SEQUENCE LISTING (239 sequences)

SEQ ID NO: 1 - DNA, 41 bp, Artificial Sequence
  Synthetic oligonucleotide P1-Adaptor (top strand)
ccactacgcctccgctttcctctctatgggcagtcggtgnt

SEQ ID NO: 2 - DNA, 42 bp, Artificial Sequence
  Synthetic oligonucleotide P1-Adaptor (bottom strand)
tcaccgactgcccatagagaggaaagcggaggcgtagtgnnc

SEQ ID NO: 3 - DNA, 41 bp, Artificial Sequence
  Synthetic oligonucleotide P1-Adaptor (top strand)
ccactacgcctccgctttcctctctatgggcagtcggtgat

SEQ ID NO: 4 - DNA, 43 bp, Artificial Sequence
  Synthetic oligonucleotide P1-Adaptor (bottom strand)
atcaccgactgcccatagagaggaaagcggaggcgtagtggtt

SEQ ID NO: 5 - DNA, 43 bp, Artificial Sequence
  Synthetic oligonucleotide P1-Adaptor (bottom strand)
atcaccgactgcccatagagaggaaagcggaggcgtagtggcc

SEQ ID NO: 6 - DNA, 25 bp, Artificial Sequence
  Synthetic oligonucleotide P2-Adaptor (top strand)
agagaatgaggaacccggggcagtt

SEQ ID NO: 7 - DNA, 23 bp, Artificial Sequence
  Synthetic oligonucleotide P2-Adaptor (bottom strand)
ctgccccgggttcctcattctct

SEQ ID NO: 8 - DNA, 25 bp, Artificial Sequence
  Synthetic oligonucleotide P2-Adaptor (top strand)
agagaatgaggaacccggggcagtt

SEQ ID NO: 9 - DNA, 25 bp, Artificial Sequence
  Synthetic oligonucleotide P2-Adaptor (top strand)
agagaatgaggaacccggggcagcc

SEQ ID NO: 10 - DNA, 23 bp, Artificial Sequence
  Synthetic oligonucleotide P2-Adaptor (bottom strand)
ctgccccgggttcctcattctct

SEQ ID NO: 11 - DNA, 24 bp, Artificial Sequence
  Synthetic oligonucleotide P2-Adaptor (top strand)
gagaatgaggaacccggggcannc

SEQ ID NO: 12 - DNA, 23 bp, Artificial Sequence
  Synthetic oligonucleotide P2-Adaptor (bottom strand)
ctgccccgggttcctcattctnt

SEQ ID NO: 13 - DNA, 28 bp, Artificial Sequence
  Synthetic oligonucleotide PCR1 primer
ccactacgcctccgctttcctctctatg

SEQ ID NO: 14 - DNA, 21 bp, Artificial Sequence
  Synthetic oligonucleotide PCR2 primer
ctgccccgggttcctcattct

SEQ ID NO: 15 - DNA, 19 bp, Artificial Sequence
  Synthetic oligonucleotide Universal adaptor
cgccttggccgtacagcag

SEQ ID NO: 16 - DNA, 53 bp, Artificial Sequence
  Synthetic oligonucleotide BC-001
ctgccccgggttcctcattctcnntgtaagaggctgctgtacggccaaggcnt

SEQ ID NO: 17 - DNA, 53 bp, Artificial Sequence
  Synthetic oligonucleotide BC-002
ctgccccgggttcctcattctcnngggagtggtctgctgtacggccaaggcnt

SEQ ID NO: 18 - DNA, 53 bp, Artificial Sequence
  Synthetic oligonucleotide BC-003
ctgccccgggttcctcattctcnntaggttatactgctgtacggccaaggcnt

SEQ ID NO: 19 - DNA, 53 bp, Artificial Sequence
  Synthetic oligonucleotide BC-004
ctgccccgggttcctcattctcnngatgcggtcctgctgtacggccaaggcnt

SEQ ID NO: 20 - DNA, 53 bp, Artificial Sequence
  Synthetic oligonucleotide BC-005
ctgccccgggttcctcattctcnntggtgtaagctgctgtacggccaaggcnt

SEQ ID NO: 21 - DNA, 53 bp, Artificial Sequence
  Synthetic oligonucleotide BC-006
ctgccccgggttcctcattctcnncgagggacactgctgtacggccaaggcnt

SEQ ID NO: 22 - DNA, 53 bp, Artificial Sequence
  Synthetic oligonucleotide BC-007
ctgccccgggttcctcattctcnnggttatgccctgctgtacggccaaggcnt

SEQ ID NO: 23 - DNA, 53 bp, Artificial Sequence
  Synthetic oligonucleotide BC-008
ctgccccgggttcctcattctcnnagcgaggatctgctgtacggccaaggcnt

SEQ ID NO: 24 - DNA, 53 bp, Artificial Sequence
  Synthetic oligonucleotide BC-009
ctgccccgggttcctcattctcnnggttgcgacctgctgtacggccaaggcnt

SEQ ID NO: 25 - DNA, 53 bp, Artificial Sequence
  Synthetic oligonucleotide BC-010
ctgccccgggttcctcattctcnncggtaagctctgctgtacggccaaggcnt

SEQ ID NO: 26 - DNA, 53 bp, Artificial Sequence
  Synthetic oligonucleotide BC-011
ctgccccgggttcctcattctcnntgcgacacgctgctgtacggccaaggcnt

SEQ ID NO: 27 - DNA, 53 bp, Artificial Sequence
  Synthetic oligonucleotide BC-012
ctgccccgggttcctcattctcnnagaggaaaactgctgtacggccaaggcnt

SEQ ID NO: 28 - DNA, 53 bp, Artificial Sequence
  Synthetic oligonucleotide BC-013
ctgccccgggttcctcattctcnncggtaaggcctgctgtacggccaaggcnt

SEQ ID NO: 29 - DNA, 53 bp, Artificial Sequence
  Synthetic oligonucleotide BC-014
ctgccccgggttcctcattctcnntgcggcagactgctgtacggccaaggcnt

SEQ ID NO: 30 - DNA, 53 bp, Artificial Sequence
  Synthetic oligonucleotide BC-015
ctgccccgggttcctcattctcnnagttgaatgctgctgtacggccaaggcnt

SEQ ID NO: 31 - DNA, 53 bp, Artificial Sequence
  Synthetic oligonucleotide BC-016
ctgccccgggttcctcattctcnnggagacgttctgctgtacggccaaggcnt

SEQ ID NO: 32 - DNA, 53 bp, Artificial Sequence
  Synthetic oligonucleotide BC-017
ctgccccgggttcctcattctcnngctcaccgcctgctgtacggccaaggcnt

SEQ ID NO: 33 - DNA, 53 bp, Artificial Sequence
  Synthetic oligonucleotide BC-018
ctgccccgggttcctcattctcnnggcggatgactgctgtacggccaaggcnt

SEQ ID NO: 34 - DNA, 53 bp, Artificial Sequence
  Synthetic oligonucleotide BC-019
ctgccccgggttcctcattctcnntggtaactgctgctgtacggccaaggcnt

SEQ ID NO: 35 - DNA, 53 bp, Artificial Sequence
  Synthetic oligonucleotide BC-020
ctgccccgggttcctcattctcnntcaagctttctgctgtacggccaaggcnt

SEQ ID NO: 36 - DNA, 53 bp, Artificial Sequence
  Synthetic oligonucleotide BC-021
ctgccccgggttcctcattctcnntgcggttccctgctgtacggccaaggcnt

SEQ ID NO: 37 - DNA, 53 bp, Artificial Sequence
  Synthetic oligonucleotide BC-022
ctgccccgggttcctcattctcnnagaagatgactgctgtacggccaaggcnt

SEQ ID NO: 38 - DNA, 53 bp, Artificial Sequence
  Synthetic oligonucleotide BC-023
ctgccccgggttcctcattctcnncggtgcttgctgctgtacggccaaggcnt

SEQ ID NO: 39 - DNA, 53 bp, Artificial Sequence
  Synthetic oligonucleotide BC-024
ctgccccgggttcctcattctcnnggtcggtatctgctgtacggccaaggcnt

SEQ ID NO: 40 - DNA, 53 bp, Artificial Sequence
  Synthetic oligonucleotide BC-025
ctgccccgggttcctcattctcnnacatgatgactgctgtacggccaaggcnt

SEQ ID NO: 41 - DNA, 53 bp, Artificial Sequence
  Synthetic oligonucleotide BC-026
ctgccccgggttcctcattctcnngggagcccgctgctgtacggccaaggcnt

SEQ ID NO: 42 - DNA, 53 bp, Artificial Sequence
  Synthetic oligonucleotide BC-027
ctgccccgggttcctcattctcnnagcaaacttctgctgtacggccaaggcnt

SEQ ID NO: 43 - DNA, 53 bp, Artificial Sequence
  Synthetic oligonucleotide BC-028
ctgccccgggttcctcattctcnngcttactacctgctgtacggccaaggcnt

SEQ ID NO: 44 - DNA, 53 bp, Artificial Sequence
  Synthetic oligonucleotide BC-029
ctgccccgggttcctcattctcnnaatctagggctgctgtacggccaaggcnt

SEQ ID NO: 45 - DNA, 53 bp, Artificial Sequence
  Synthetic oligonucleotide BC-030
ctgccccgggttcctcattctcnntagcgaagactgctgtacggccaaggcnt

SEQ ID NO: 46 - DNA, 53 bp, Artificial Sequence
  Synthetic oligonucleotide BC-031
ctgccccgggttcctcattctcnnctggtgcgtctgctgtacggccaaggcnt

SEQ ID NO: 47 - DNA, 53 bp, Artificial Sequence
  Synthetic oligonucleotide BC-032
ctgccccgggttcctcattctcnngttgggtgcctgctgtacggccaaggcnt

SEQ ID NO: 48 - DNA, 53 bp, Artificial Sequence
  Synthetic oligonucleotide BC-033
ctgccccgggttcctcattctcnngttggatacctgctgtacggccaaggcnt

SEQ ID NO: 49 - DNA, 53 bp, Artificial Sequence
  Synthetic oligonucleotide BC-034
ctgccccgggttcctcattctcnncgttaaaggctgctgtacggccaaggcnt

SEQ ID NO: 50 - DNA, 53 bp, Artificial Sequence
  Synthetic oligonucleotide BC-035
ctgccccgggttcctcattctcnnagcgtaggactgctgtacggccaaggcnt

SEQ ID NO: 51 - DNA, 53 bp, Artificial Sequence
  Synthetic oligonucleotide BC-036
ctgccccgggttcctcattctcnnttctcacatctgctgtacggccaaggcnt

SEQ ID NO: 52 - DNA, 53 bp, Artificial Sequence
  Synthetic oligonucleotide BC-037
ctgccccgggttcctcattctcnntgttataccctgctgtacggccaaggcnt

SEQ ID NO: 53 - DNA, 53 bp, Artificial Sequence
  Synthetic oligonucleotide BC-038
ctgccccgggttcctcattctcnntcgtcttagctgctgtacggccaaggcnt

SEQ ID NO: 54 - DNA, 53 bp, Artificial Sequence
  Synthetic oligonucleotide BC-039
ctgccccgggttcctcattctcnnatcgtgagtctgctgtacggccaaggcnt

SEQ ID NO: 55 - DNA, 53 bp, Artificial Sequence
  Synthetic oligonucleotide BC-040
ctgccccgggttcctcattctcnnaaagggttactgctgtacggccaaggcnt

SEQ ID NO: 56 - DNA, 53 bp, Artificial Sequence
  Synthetic oligonucleotide BC-041
ctgccccgggttcctcattctcnngtgggattgctgctgtacggccaaggcnt

SEQ ID NO: 57 - DNA, 53 bp, Artificial Sequence
  Synthetic oligonucleotide BC-042
ctgccccgggttcctcattctcnnaatgtactactgctgtacggccaaggcnt

SEQ ID NO: 58 - DNA, 53 bp, Artificial Sequence
  Synthetic oligonucleotide BC-043
ctgccccgggttcctcattctcnngctagggttctgctgtacggccaaggcnt

SEQ ID NO: 59 - DNA, 53 bp, Artificial Sequence
  Synthetic oligonucleotide BC-044
ctgccccgggttcctcattctcnnaggatgatcctgctgtacggccaaggcnt

SEQ ID NO: 60 - DNA, 53 bp, Artificial Sequence
  Synthetic oligonucleotide BC-045
ctgccccgggttcctcattctcnntacttggctctgctgtacggccaaggcnt

SEQ ID NO: 61 - DNA, 53 bp, Artificial Sequence
  Synthetic oligonucleotide BC-046
ctgccccgggttcctcattctcnngtcgtcgaactgctgtacggccaaggcnt

SEQ ID NO: 62 - DNA, 53 bp, Artificial Sequence
  Synthetic oligonucleotide BC-047
ctgccccgggttcctcattctcnnagggatggcctgctgtacggccaaggcnt

SEQ ID NO: 63 - DNA, 53 bp, Artificial Sequence
  Synthetic oligonucleotide BC-048
ctgccccgggttcctcattctcnnccgtaagtgctgctgtacggccaaggcnt

SEQ ID NO: 64 - DNA, 53 bp, Artificial Sequence
  Synthetic oligonucleotide BC-049
ctgccccgggttcctcattctcnntgtcataagctgctgtacggccaaggcnt

SEQ ID NO: 65 - DNA, 53 bp, Artificial Sequence
  Synthetic oligonucleotide BC-050
ctgccccgggttcctcattctcnnaaggcttgcctgctgtacggccaaggcnt

SEQ ID NO: 66 - DNA, 53 bp, Artificial Sequence
  Synthetic oligonucleotide BC-051
ctgccccgggttcctcattctcnnagcaggagtctgctgtacggccaaggcnt

SEQ ID NO: 67 - DNA, 53 bp, Artificial Sequence
  Synthetic oligonucleotide BC-052
ctgccccgggttcctcattctcnntaattgtaactgctgtacggccaaggcnt

SEQ ID NO: 68 - DNA, 53 bp, Artificial Sequence
  Synthetic oligonucleotide BC-053
ctgccccgggttcctcattctcnntcatcaagtctgctgtacggccaaggcnt

SEQ ID NO: 69 - DNA, 53 bp, Artificial Sequence
  Synthetic oligonucleotide BC-054
ctgccccgggttcctcattctcnnaaaggcggactgctgtacggccaaggcnt

SEQ ID NO: 70 - DNA, 53 bp, Artificial Sequence
  Synthetic oligonucleotide BC-055
ctgccccgggttcctcattctcnngcttaagcgctgctgtacggccaaggcnt

SEQ ID NO: 71 - DNA, 53 bp, Artificial Sequence
  Synthetic oligonucleotide BC-056
ctgccccgggttcctcattctcnncatgtcaccctgctgtacggccaaggcnt

SEQ ID NO: 72 - DNA, 53 bp, Artificial Sequence
  Synthetic oligonucleotide BC-057
ctgccccgggttcctcattctcnntagtaagaactgctgtacggccaaggcnt

SEQ ID NO: 73 - DNA, 53 bp, Artificial Sequence
  Synthetic oligonucleotide BC-058
ctgccccgggttcctcattctcnnaaagtggcgctgctgtacggccaaggcnt

SEQ ID NO: 74 - DNA, 53 bp, Artificial Sequence
  Synthetic oligonucleotide BC-059
ctgccccgggttcctcattctcnnagtaatgtcctgctgtacggccaaggcnt

SEQ ID NO: 75 - DNA, 53 bp, Artificial Sequence
  Synthetic oligonucleotide BC-060
ctgccccgggttcctcattctcnntgcctcggtctgctgtacggccaaggcnt

SEQ ID NO: 76 - DNA, 53 bp, Artificial Sequence
  Synthetic oligonucleotide BC-061
ctgccccgggttcctcattctcnnagattatcgctgctgtacggccaaggcnt

SEQ ID NO: 77 - DNA, 53 bp, Artificial Sequence
  Synthetic oligonucleotide BC-062
ctgccccgggttcctcattctcnnggtgagggtctgctgtacggccaaggcnt

SEQ ID NO: 78 - DNA, 53 bp, Artificial Sequence
  Synthetic oligonucleotide BC-063
ctgccccgggttcctcattctcnncgggttcgactgctgtacggccaaggcnt

SEQ ID NO: 79 - DNA, 53 bp, Artificial Sequence
  Synthetic oligonucleotide BC-064
ctgccccgggttcctcattctcnntgctacaccctgctgtacggccaaggcnt

SEQ ID NO: 80 - DNA, 53 bp, Artificial Sequence
  Synthetic oligonucleotide BC-065
ctgccccgggttcctcattctcnnggatcaagcctgctgtacggccaaggcnt

SEQ ID NO: 81 - DNA, 53 bp, Artificial Sequence
  Synthetic oligonucleotide BC-066
ctgccccgggttcctcattctcnnatgtaatgtctgctgtacggccaaggcnt

SEQ ID NO: 82 - DNA, 53 bp, Artificial Sequence
  Synthetic oligonucleotide BC-067
ctgccccgggttcctcattctcnntccttagggctgctgtacggccaaggcnt

SEQ ID NO: 83 - DNA, 53 bp, Artificial Sequence
  Synthetic oligonucleotide BC-068
ctgccccgggttcctcattctcnncattgacgactgctgtacggccaaggcnt

SEQ ID NO: 84 - DNA, 53 bp, Artificial Sequence
  Synthetic oligonucleotide BC-069
ctgccccgggttcctcattctcnnatatgctttctgctgtacggccaaggcnt

SEQ ID NO: 85 - DNA, 53 bp, Artificial Sequence
  Synthetic oligonucleotide BC-070
ctgccccgggttcctcattctcnnccctacagactgctgtacggccaaggcnt

SEQ ID NO: 86 - DNA, 53 bp, Artificial Sequence
  Synthetic oligonucleotide BC-071
ctgccccgggttcctcattctcnncagggaacgctgctgtacggccaaggcnt

SEQ ID NO: 87 - DNA, 53 bp, Artificial Sequence
  Synthetic oligonucleotide BC-072
ctgccccgggttcctcattctcnnagtgaatacctgctgtacggccaaggcnt

SEQ ID NO: 88 - DNA, 53 bp, Artificial Sequence
  Synthetic oligonucleotide BC-073
ctgccccgggttcctcattctcnncaatgacgtctgctgtacggccaaggcnt

SEQ ID NO: 89 - DNA, 53 bp, Artificial Sequence
  Synthetic oligonucleotide BC-074
ctgccccgggttcctcattctcnnggacgctgactgctgtacggccaaggcnt

SEQ ID NO: 90 - DNA, 53 bp, Artificial Sequence
  Synthetic oligonucleotide BC-075
ctgccccgggttcctcattctcnntatctgggcctgctgtacggccaaggcnt

SEQ ID NO: 91 - DNA, 53 bp, Artificial Sequence
  Synthetic oligonucleotide BC-076
ctgccccgggttcctcattctcnnagttttaggctgctgtacggccaaggcnt

SEQ ID NO: 92 - DNA, 53 bp, Artificial Sequence
  Synthetic oligonucleotide BC-077
ctgccccgggttcctcattctcnntctggtcttctgctgtacggccaaggcnt

SEQ ID NO: 93 - DNA, 53 bp, Artificial Sequence
  Synthetic oligonucleotide BC-078
ctgccccgggttcctcattctcnngcaatcatcctgctgtacggccaaggcnt

SEQ ID NO: 94 - DNA, 53 bp, Artificial Sequence
  Synthetic oligonucleotide BC-079
ctgccccgggttcctcattctcnngtagaattactgctgtacggccaaggcnt

SEQ ID NO: 95 - DNA, 53 bp, Artificial Sequence
  Synthetic oligonucleotide BC-080
ctgccccgggttcctcattctcnntttacggtgctgctgtacggccaaggcnt

SEQ ID NO: 96 - DNA, 53 bp, Artificial Sequence
  Synthetic oligonucleotide BC-081
ctgccccgggttcctcattctcnnaacgtcattctgctgtacggccaaggcnt

SEQ ID NO: 97 - DNA, 53 bp, Artificial Sequence
  Synthetic oligonucleotide BC-082
ctgccccgggttcctcattctcnntgaagggagctgctgtacggccaaggcnt

SEQ ID NO: 98 - DNA, 53 bp, Artificial Sequence
  Synthetic oligonucleotide BC-083
ctgccccgggttcctcattctcnngatggcgtactgctgtacggccaaggcnt

SEQ ID NO: 99 - DNA, 53 bp, Artificial Sequence
  Synthetic oligonucleotide BC-084
ctgccccgggttcctcattctcnncggatgaacctgctgtacggccaaggcnt

SEQ ID NO: 100 - DNA, 53 bp, Artificial Sequence
  Synthetic oligonucleotide BC-085
ctgccccgggttcctcattctcnngaaagcgttctgctgtacggccaaggcnt

SEQ ID NO: 101 - DNA, 53 bp, Artificial Sequence
  Synthetic oligonucleotide BC-086
ctgccccgggttcctcattctcnngtaccaggactgctgtacggccaaggcnt

SEQ ID NO: 102 - DNA, 53 bp, Artificial Sequence
  Synthetic oligonucleotide BC-087
ctgccccgggttcctcattctcnntagcaaagcctgctgtacggccaaggcnt

SEQ ID NO: 103 - DNA, 53 bp, Artificial Sequence
  Synthetic oligonucleotide BC-088
ctgccccgggttcctcattctcnnttgatcatgctgctgtacggccaaggcnt

SEQ ID NO: 104 - DNA, 53 bp, Artificial Sequence
  Synthetic oligonucleotide BC-089
ctgccccgggttcctcattctcnnggctgtctactgctgtacggccaaggcnt

SEQ ID NO: 105 - DNA, 53 bp, Artificial Sequence
  Synthetic oligonucleotide BC-090
ctgccccgggttcctcattctcnntgacctactctgctgtacggccaaggcnt

SEQ ID NO: 106 - DNA, 53 bp, Artificial Sequence
  Synthetic oligonucleotide BC-091
ctgccccgggttcctcattctcnncgtattgggctgctgtacggccaaggcnt

SEQ ID NO: 107 - DNA, 53 bp, Artificial Sequence
  Synthetic oligonucleotide BC-092
ctgccccgggttcctcattctcnnagggattacctgctgtacggccaaggcnt

SEQ ID NO: 108 - DNA, 53 bp, Artificial Sequence
  Synthetic oligonucleotide BC-093
ctgccccgggttcctcattctcnnttacgatgcctgctgtacggccaaggcnt

SEQ ID NO: 109 - DNA, 53 bp, Artificial Sequence
  Synthetic oligonucleotide BC-094
ctgccccgggttcctcattctcnntgggtgtttctgctgtacggccaaggcnt

SEQ ID NO: 110 - DNA, 53 bp, Artificial Sequence
  Synthetic oligonucleotide BC-095
ctgccccgggttcctcattctcnnagtccggcactgctgtacggccaaggcnt

SEQ ID NO: 111 - DNA, 53 bp, Artificial Sequence
  Synthetic oligonucleotide BC-096
ctgccccgggttcctcattctcnnatcgaagagctgctgtacggccaaggcnt

SEQ ID NO: 112 - DNA, 41 bp, Artificial Sequence
  Synthetic oligonucleotide P1 Blocker
atcaccgactgcccatagagaggaaagcggaggcgtagtgg

SEQ ID NO: 113 - DNA, 52 bp, Artificial Sequence
  Synthetic oligonucleotide Barcode-001 Blocker
cgccttggccgtacagcagcctcttacacagagaatgaggaacccggggcag

SEQ ID NO: 114 - DNA, 52 bp, Artificial Sequence
  Synthetic oligonucleotide Barcode-002 Blocker
cgccttggccgtacagcagaccactccctagagaatgaggaacccggggcag

SEQ ID NO: 115 - DNA, 52 bp, Artificial Sequence
  Synthetic oligonucleotide Barcode-003 Blocker
cgccttggccgtacagcagtataacctatagagaatgaggaacccggggcag

SEQ ID NO: 116 - DNA, 52 bp, Artificial Sequence
  Synthetic oligonucleotide Barcode-004 Blocker
cgccttggccgtacagcaggaccgcatccagagaatgaggaacccggggcag

SEQ ID NO: 117 - DNA, 52 bp, Artificial Sequence
  Synthetic oligonucleotide Barcode-005 Blocker
cgccttggccgtacagcagcttacaccacagagaatgaggaacccggggcag

SEQ ID NO: 118 - DNA, 52 bp, Artificial Sequence
  Synthetic oligonucleotide Barcode-006 Blocker
cgccttggccgtacagcagtgtccctcgcagagaatgaggaacccggggcag

SEQ ID NO: 119 - DNA, 52 bp, Artificial Sequence
  Synthetic oligonucleotide Barcode-007 Blocker
cgccttggccgtacagcagggcataacccagagaatgaggaacccggggcag

SEQ ID NO: 120 - DNA, 52 bp, Artificial Sequence
  Synthetic oligonucleotide Barcode-008 Blocker
cgccttggccgtacagcagatcctcgctcagagaatgaggaacccggggcag

SEQ ID NO: 121 - DNA, 52 bp, Artificial Sequence
  Synthetic oligonucleotide Barcode-009 Blocker
cgccttggccgtacagcaggtcgcaacctagagaatgaggaacccggggcag

SEQ ID NO: 122 - DNA, 52 bp, Artificial Sequence
  Synthetic oligonucleotide Barcode-010 Blocker
cgccttggccgtacagcagagcttaccgcagagaatgaggaacccggggcag

SEQ ID NO: 123 - DNA, 52 bp, Artificial Sequence
  Synthetic oligonucleotide Barcode-011 Blocker
cgccttggccgtacagcagcgtgtcgcacagagaatgaggaacccggggcag

SEQ ID NO: 124 - DNA, 52 bp, Artificial Sequence
  Synthetic oligonucleotide Barcode-012 Blocker
cgccttggccgtacagcagttttcctcttagagaatgaggaacccggggcag

SEQ ID NO: 125 - DNA, 52 bp, Artificial Sequence
  Synthetic oligonucleotide Barcode-013 Blocker
cgccttggccgtacagcaggccttaccgcagagaatgaggaacccggggcag

SEQ ID NO: 126 - DNA, 52 bp, Artificial Sequence
  Synthetic oligonucleotide Barcode-014 Blocker
cgccttggccgtacagcagtctgccgcacagagaatgaggaacccggggcag

SEQ ID NO: 127 - DNA, 52 bp, Artificial Sequence
  Synthetic oligonucleotide Barcode-015 Blocker
cgccttggccgtacagcagcattcaactcagagaatgaggaacccggggcag

SEQ ID NO: 128 - DNA, 52 bp, Artificial Sequence
  Synthetic oligonucleotide Barcode-016 Blocker
cgccttggccgtacagcagaacgtctcccagagaatgaggaacccggggcag

SEQ ID NO: 129 - DNA, 41 bp, Artificial Sequence; identical to SEQ ID NO: 3
  Synthetic oligonucleotide P1-Adaptor (top strand)

SEQ ID NO: 130 - DNA, 43 bp, Artificial Sequence
  Synthetic oligonucleotide P1-Adaptor (bottom strand)
atcaccgactgcccatagagaggaaagcggaggcgtagtggnn

SEQ ID NO: 131 - DNA, 28 bp, Artificial Sequence
  Synthetic oligonucleotide A adapter (top strand)
gtcggagacacgcagggatgagatggnn

SEQ ID NO: 132 - DNA, 26 bp, Artificial Sequence
  Synthetic oligonucleotide A adapter (bottom strand)
ccatctcatccctgcgtgtctccgac

SEQ ID NO: 133 - DNA, 32 bp, Artificial Sequence
  Synthetic oligonucleotide Barcoded A adapter (top strand)
nnnngtcggagacacgcagggatgagatggnn

SEQ ID NO: 134 - DNA, 43 bp, Artificial Sequence
  Synthetic oligonucleotide Barcoded A adapter (bottom strand)
ccatctcatccctgcgtgtctccgactcagnnnnnnnnnnagt

SEQ ID NO: 135 - DNA, 30 bp, Artificial Sequence
  Synthetic oligonucleotide Blocking oligo 1
actnnnnnnnnnnctgagtcggagacacgc

SEQ ID NO: 136 - DNA, 43 bp, Artificial Sequence
  Synthetic oligonucleotide Blocking oligo 2
atcnnnnnnnnnnctgagtcggagacacgcagggatgagatgg

SEQ ID NO: 137 - DNA, 17 bp, Artificial Sequence
  Synthetic oligonucleotide Blocking oligo 3
ctgagtcggagacacgc

SEQ ID NO: 138 - DNA, 30 bp, Artificial Sequence
  Synthetic oligonucleotide Blocking oligo 4
ctgagtcggagacacgcagggatgagatgg

SEQ ID NO: 139 - DNA, 30 bp, Artificial Sequence; identical to SEQ ID NO: 138
  Synthetic oligonucleotide A Blocker

SEQ ID NO: 140 - DNA, 32 bp, Artificial Sequence
  Synthetic oligonucleotide A adapter (top strand)
ttccatctcatccctgcgtgtctccgactcag

SEQ ID NO: 141 - DNA, 34 bp, Artificial Sequence
  Synthetic oligonucleotide A adapter (bottom strand)
ctgagtcggagacacgcagggatgagatggaatt

SEQ ID NO: 142 - DNA, 30 bp, Artificial Sequence; identical to SEQ ID NO: 138
  Synthetic oligonucleotide Blocker A

SEQ ID NO: 143 - DNA, 41 bp, Artificial Sequence; identical to SEQ ID NO: 112
  Synthetic oligonucleotide Blocker P1

SEQ ID NO: 144 - DNA, 43 bp, Artificial Sequence
  Synthetic oligonucleotide Blocker BC 1
atcgttaccttagctgagtcggagacacgcagggatgagatgg

SEQ ID NO: 145 - DNA, 43 bp, Artificial Sequence
  Synthetic oligonucleotide Blocker BC 2
atcgttctccttactgagtcggagacacgcagggatgagatgg

SEQ ID NO: 146 - DNA, 43 bp, Artificial Sequence
  Synthetic oligonucleotide Blocker BC 3
atcgaatcctcttctgagtcggagacacgcagggatgagatgg

SEQ ID NO: 147 - DNA, 43 bp, Artificial Sequence
  Synthetic oligonucleotide Blocker BC 4
atcgatcttggtactgagtcggagacacgcagggatgagatgg

SEQ ID NO: 148 - DNA, 43 bp, Artificial Sequence
  Synthetic oligonucleotide Blocker BC 5
atcgttccttctgctgagtcggagacacgcagggatgagatgg

SEQ ID NO: 149 - DNA, 43 bp, Artificial Sequence
  Synthetic oligonucleotide Blocker BC 6
atcgaacttgcagctgagtcggagacacgcagggatgagatgg

SEQ ID NO: 150 - DNA, 43 bp, Artificial Sequence
  Synthetic oligonucleotide Blocker BC 7
atcgaatcacgaactgagtcggagacacgcagggatgagatgg

SEQ ID NO: 151 - DNA, 43 bp, Artificial Sequence
  Synthetic oligonucleotide Blocker BC 8
atcgttatcggaactgagtcggagacacgcagggatgagatgg

SEQ ID NO: 152 - DNA, 43 bp, Artificial Sequence
  Synthetic oligonucleotide Blocker BC 9
atcgttccgctcactgagtcggagacacgcagggatgagatgg

SEQ ID NO: 153 - DNA, 43 bp, Artificial Sequence
  Synthetic oligonucleotide Blocker BC 10
atcgttcggtcagctgagtcggagacacgcagggatgagatgg

SEQ ID NO: 154 - DNA, 43 bp, Artificial Sequence
  Synthetic oligonucleotide Blocker BC 11
atcgattcgaggactgagtcggagacacgcagggatgagatgg

SEQ ID NO: 155 - DNA, 43 bp, Artificial Sequence
  Synthetic oligonucleotide Blocker BC 12
atcgaaccacctactgagtcggagacacgcagggatgagatgg

SEQ ID NO: 156 - DNA, 43 bp, Artificial Sequence
  Synthetic oligonucleotide Blocker BC 13
atcgtccgttagactgagtcggagacacgcagggatgagatgg

SEQ ID NO: 157 - DNA, 43 bp, Artificial Sequence
  Synthetic oligonucleotide Blocker BC 14
atcgacactccaactgagtcggagacacgcagggatgagatgg

SEQ ID NO: 158 - DNA, 43 bp, Artificial Sequence
  Synthetic oligonucleotide Blocker BC 15
atcgacctctagactgagtcggagacacgcagggatgagatgg

SEQ ID NO: 159 - DNA, 43 bp, Artificial Sequence
  Synthetic oligonucleotide Blocker BC 16
atcgtcatccagactgagtcggagacacgcagggatgagatgg

SEQ ID NO: 160 - DNA, 43 bp, Artificial Sequence
  Synthetic oligonucleotide Blocker BC 17
atcgacgaatagactgagtcggagacacgcagggatgagatgg

SEQ ID NO: 161 - DNA, 43 bp, Artificial Sequence
  Synthetic oligonucleotide Blocker BC 18
atcgcaattgcctctgagtcggagacacgcagggatgagatgg

SEQ ID NO: 162 - DNA, 43 bp, Artificial Sequence
  Synthetic oligonucleotide Blocker BC 19
atcgtccgactaactgagtcggagacacgcagggatgagatgg

SEQ ID NO: 163 - DNA, 43 bp, Artificial Sequence
  Synthetic oligonucleotide Blocker BC 20
atcgatggatctgctgagtcggagacacgcagggatgagatgg

SEQ ID NO: 164 - DNA, 43 bp, Artificial Sequence
  Synthetic oligonucleotide Blocker BC 21
atcgtaattgcgactgagtcggagacacgcagggatgagatgg

SEQ ID NO: 165 - DNA, 43 bp, Artificial Sequence
  Synthetic oligonucleotide Blocker BC 22
atcgcgtctcgaactgagtcggagacacgcagggatgagatgg

SEQ ID NO: 166 - DNA, 43 bp, Artificial Sequence
  Synthetic oligonucleotide Blocker BC 23
atcgttcgtggcactgagtcggagacacgcagggatgagatgg

SEQ ID NO: 167 - DNA, 43 bp, Artificial Sequence
  Synthetic oligonucleotide Blocker BC 24
atcgaatgaggttctgagtcggagacacgcagggatgagatgg

SEQ ID NO: 168 - DNA, 43 bp, Artificial Sequence
  Synthetic oligonucleotide Blocker BC 25
atcgtatctcaggctgagtcggagacacgcagggatgagatgg

SEQ ID NO: 169 - DNA, 43 bp, Artificial Sequence
  Synthetic oligonucleotide Blocker BC 26
atcgaggttgtaactgagtcggagacacgcagggatgagatgg

SEQ ID NO: 170 - DNA, 43 bp, Artificial Sequence
  Synthetic oligonucleotide Blocker BC 27
atcgcggatggttctgagtcggagacacgcagggatgagatgg

SEQ ID NO: 171 - DNA, 43 bp, Artificial Sequence
  Synthetic oligonucleotide Blocker BC 28
atcgattccggatctgagtcggagacacgcagggatgagatgg

SEQ ID NO: 172 - DNA, 43 bp, Artificial Sequence
  Synthetic oligonucleotide Blocker BC 29
atcgagtggtcgactgagtcggagacacgcagggatgagatgg

SEQ ID NO: 173 - DNA, 43 bp, Artificial Sequence
  Synthetic oligonucleotide Blocker BC 30
atcgataacctcgctgagtcggagacacgcagggatgagatgg

SEQ ID NO: 174 - DNA, 43 bp, Artificial Sequence
  Synthetic oligonucleotide Blocker BC 31
atcgcagcttggactgagtcggagacacgcagggatgagatgg

SEQ ID NO: 175 - DNA, 43 bp, Artificial Sequence
  Synthetic oligonucleotide Blocker BC 32
atcgtgtgtaagactgagtcggagacacgcagggatgagatgg

SEQ ID NO: 176 - DNA, 45 bp, Artificial Sequence
  Synthetic oligonucleotide Blocker BC 33
atcgttcaatgagaactgagtcggagacacgcagggatgagatgg

SEQ ID NO: 177 - DNA, 44 bp, Artificial Sequence
  Synthetic oligonucleotide Blocker BC 34
atcgaacgatgcgactgagtcggagacacgcagggatgagatgg

SEQ ID NO: 178 - DNA, 45 bp, Artificial Sequence
  Synthetic oligonucleotide Blocker BC 35
atcgacaatggcttactgagtcggagacacgcagggatgagatgg

SEQ ID NO: 179 - DNA, 44 bp, Artificial Sequence
  Synthetic oligonucleotide Blocker BC 36
atcgacgattccttctgagtcggagacacgcagggatgagatgg

SEQ ID NO: 180 - DNA, 45 bp, Artificial Sequence
  Synthetic oligonucleotide Blocker BC 37
atcgacattctcaagctgagtcggagacacgcagggatgagatgg

SEQ ID NO: 181 - DNA, 45 bp, Artificial Sequence
  Synthetic oligonucleotide Blocker BC 38
atcgtccgtcctccactgagtcggagacacgcagggatgagatgg

SEQ ID NO: 182 - DNA, 44 bp, Artificial Sequence
  Synthetic oligonucleotide Blocker BC 39
atcgccgattgttactgagtcggagacacgcagggatgagatgg

SEQ ID NO: 183 - DNA, 44 bp, Artificial Sequence
  Synthetic oligonucleotide Blocker BC 40
atcgattatgtcagctgagtcggagacacgcagggatgagatgg

SEQ ID NO: 184 - DNA, 44 bp, Artificial Sequence
  Synthetic oligonucleotide Blocker BC 41
atcgcgaagtggaactgagtcggagacacgcagggatgagatgg

SEQ ID NO: 185 - DNA, 43 bp, Artificial Sequence
  Synthetic oligonucleotide Blocker BC 42
atcgattcgtgctctgagtcggagacacgcagggatgagatgg

SEQ ID NO: 186 - DNA, 44 bp, Artificial Sequence
  Synthetic oligonucleotide Blocker BC 43
atcgcggtgtcaagctgagtcggagacacgcagggatgagatgg

SEQ ID NO: 187 - DNA, 45 bp, Artificial Sequence
  Synthetic oligonucleotide Blocker BC 44
atcgctggcctccaactgagtcggagacacgcagggatgagatgg

SEQ ID NO: 188 - DNA, 45 bp, Artificial Sequence
  Synthetic oligonucleotide Blocker BC 45
atcgaggaagctccactgagtcggagacacgcagggatgagatgg

SEQ ID NO: 189 - DNA, 44 bp, Artificial Sequence
  Synthetic oligonucleotide Blocker BC 46
atcgttcggactgactgagtcggagacacgcagggatgagatgg

SEQ ID NO: 190 - DNA, 45 bp, Artificial Sequence
  Synthetic oligonucleotide Blocker BC 47
atcgtggttgccttactgagtcggagacacgcagggatgagatgg

SEQ ID NO: 191 - DNA, 44 bp, Artificial Sequence
  Synthetic oligonucleotide Blocker BC 48
atcgtctcttagaactgagtcggagacacgcagggatgagatgg

SEQ ID NO: 192 - DNA, 45 bp, Artificial Sequence
  Synthetic oligonucleotide Blocker BC 49
atcgttatgttaggactgagtcggagacacgcagggatgagatgg

SEQ ID NO: 193 - DNA, 44 bp, Artificial Sequence
  Synthetic oligonucleotide Blocker BC 50
atcgccattgtccgctgagtcggagacacgcagggatgagatgg

SEQ ID NO: 194 - DNA, 45 bp, Artificial Sequence
  Synthetic oligonucleotide Blocker BC 51
atcgaataggctcaactgagtcggagacacgcagggatgagatgg

SEQ ID NO: 195 - DNA, 44 bp, Artificial Sequence
  Synthetic oligonucleotide Blocker BC 52
atcgttccatgcggctgagtcggagacacgcagggatgagatgg

SEQ ID NO: 196 - DNA, 45 bp, Artificial Sequence
  Synthetic oligonucleotide Blocker BC 53
atcgaggattgccagctgagtcggagacacgcagggatgagatgg

SEQ ID NO: 197 - DNA, 45 bp, Artificial Sequence
  Synthetic oligonucleotide Blocker BC 54
atcgcgattctccggctgagtcggagacacgcagggatgagatgg

SEQ ID NO: 198 - DNA, 44 bp, Artificial Sequence
  Synthetic oligonucleotide Blocker BC 55
atcgaggaggtggactgagtcggagacacgcagggatgagatgg

SEQ ID NO: 199 - DNA, 45 bp, Artificial Sequence
  Synthetic oligonucleotide Blocker BC 56
atcgaattaatgctgctgagtcggagacacgcagggatgagatgg

SEQ ID NO: 200 - DNA, 45 bp, Artificial Sequence
  Synthetic oligonucleotide Blocker BC 57
atcgccgttgccagactgagtcggagacacgcagggatgagatgg

SEQ ID NO: 201 - DNA, 44 bp, Artificial Sequence
  Synthetic oligonucleotide Blocker BC 58
atcgtgttctaggactgagtcggagacacgcagggatgagatgg

SEQ ID NO: 202 - DNA, 45 bp, Artificial Sequence
  Synthetic oligonucleotide Blocker BC 59
atcgaacatcaaggactgagtcggagacacgcagggatgagatgg

SEQ ID NO: 203 - DNA, 44 bp, Artificial Sequence
  Synthetic oligonucleotide Blocker BC 60
atcgaagagctagactgagtcggagacacgcagggatgagatgg

SEQ ID NO: 204 - DNA, 44 bp, Artificial Sequence
  Synthetic oligonucleotide Blocker BC 61
atcgatccgagtgactgagtcggagacacgcagggatgagatgg

SEQ ID NO: 205 - DNA, 45 bp, Artificial Sequence
  Synthetic oligonucleotide Blocker BC 62
atcgtgaagcaggaactgagtcggagacacgcagggatgagatgg

SEQ ID NO: 206 - DNA, 44 bp, Artificial Sequence
  Synthetic oligonucleotide Blocker BC 63
atcgaactctaaggctgagtcggagacacgcagggatgagatgg

SEQ ID NO: 207 - DNA, 45 bp, Artificial Sequence
  Synthetic oligonucleotide Blocker BC 64
atcgtcggaactcagctgagtcggagacacgcagggatgagatgg

SEQ ID NO: 208 - DNA, 45 bp, Artificial Sequence
  Synthetic oligonucleotide Blocker BC 65
atcgatgtgccaggactgagtcggagacacgcagggatgagatgg

SEQ ID NO: 209 - DNA, 44 bp, Artificial Sequence
  Synthetic oligonucleotide Blocker BC 66
atcgatgattgcggctgagtcggagacacgcagggatgagatgg

SEQ ID NO: 210 - DNA, 45 bp, Artificial Sequence
  Synthetic oligonucleotide Blocker BC 67
atcgactggtaggaactgagtcggagacacgcagggatgagatgg

SEQ ID NO: 211 - DNA, 44 bp, Artificial Sequence
  Synthetic oligonucleotide Blocker BC 68
atcgaacttcttgactgagtcggagacacgcagggatgagatgg

SEQ ID NO: 212 - DNA, 43 bp, Artificial Sequence
  Synthetic oligonucleotide Blocker BC 69
atcgccaattgaactgagtcggagacacgcagggatgagatgg

SEQ ID NO: 213 - DNA, 43 bp, Artificial Sequence
  Synthetic oligonucleotide Blocker BC 70
atcgaccagtaggctgagtcggagacacgcagggatgagatgg

SEQ ID NO: 214 - DNA, 45 bp, Artificial Sequence
  Synthetic oligonucleotide Blocker BC 71
atcgtcggagcctcactgagtcggagacacgcagggatgagatgg

SEQ ID NO: 215 - DNA, 45 bp, Artificial Sequence
  Synthetic oligonucleotide Blocker BC 72
atcgtgtggccttcgctgagtcggagacacgcagggatgagatgg

SEQ ID NO: 216 - DNA, 43 bp, Artificial Sequence
  Synthetic oligonucleotide Blocker BC 73
atcgacaggcagactgagtcggagacacgcagggatgagatgg

SEQ ID NO: 217 - DNA, 43 bp, Artificial Sequence
  Synthetic oligonucleotide Blocker BC 74
atcgaaccgatcgctgagtcggagacacgcagggatgagatgg

SEQ ID NO: 218 - DNA, 43 bp, Artificial Sequence
  Synthetic oligonucleotide Blocker BC 75
atcgtattcctgactgagtcggagacacgcagggatgagatgg

SEQ ID NO: 219 - DNA, 45 bp, Artificial Sequence
  Synthetic oligonucleotide Blocker BC 76
atcgaggttcttccgctgagtcggagacacgcagggatgagatgg

SEQ ID NO: 220 - DNA, 44 bp, Artificial Sequence
  Synthetic oligonucleotide Blocker BC 77
atcgaatcgcttcgctgagtcggagacacgcagggatgagatgg

SEQ ID NO: 221 - DNA, 45 bp, Artificial Sequence
  Synthetic oligonucleotide Blocker BC 78
atcgagaattggctgctgagtcggagacacgcagggatgagatgg

SEQ ID NO: 222 - DNA, 43 bp, Artificial Sequence
  Synthetic oligonucleotide Blocker BC 79
atcgacaaccaggctgagtcggagacacgcagggatgagatgg

SEQ ID NO: 223 - DNA, 45 bp, Artificial Sequence
  Synthetic oligonucleotide Blocker BC 80
atcgcctgccttcgactgagtcggagacacgcagggatgagatgg

SEQ ID NO: 224 - DNA, 45 bp, Artificial Sequence
  Synthetic oligonucleotide Blocker BC 81
atcgcgaatggcaggctgagtcggagacacgcagggatgagatgg

SEQ ID NO: 225 - DNA, 43 bp, Artificial Sequence
  Synthetic oligonucleotide Blocker BC 82
atcgagatgccaactgagtcggagacacgcagggatgagatgg

SEQ ID NO: 226 - DNA, 44 bp, Artificial Sequence
  Synthetic oligonucleotide Blocker BC 83
atcgaatgtcctagctgagtcggagacacgcagggatgagatgg

SEQ ID NO: 227 - DNA, 43 bp, Artificial Sequence
  Synthetic oligonucleotide Blocker BC 84
atcgttatggaagctgagtcggagacacgcagggatgagatgg

SEQ ID NO: 228 - DNA, 44 bp, Artificial Sequence
  Synthetic oligonucleotide Blocker BC 85
atcgttgaggctggctgagtcggagacacgcagggatgagatgg

SEQ ID NO: 229 - DNA, 44 bp, Artificial Sequence
  Synthetic oligonucleotide Blocker BC 86
atcgaataaccaagctgagtcggagacacgcagggatgagatgg

SEQ ID NO: 230 - DNA, 43 bp, Artificial Sequence
  Synthetic oligonucleotide Blocker BC 87
atcgtccagccaactgagtcggagacacgcagggatgagatgg

SEQ ID NO: 231 - DNA, 44 bp, Artificial Sequence
  Synthetic oligonucleotide Blocker BC 88
atcgaagtgttcggctgagtcggagacacgcagggatgagatgg

SEQ ID NO: 232 - DNA, 44 bp, Artificial Sequence
  Synthetic oligonucleotide Blocker BC 89
atcgagattcaggactgagtcggagacacgcagggatgagatgg

SEQ ID NO: 233 - DNA, 44 bp, Artificial Sequence
  Synthetic oligonucleotide Blocker BC 90
atcgccgtggttagctgagtcggagacacgcagggatgagatgg

SEQ ID NO: 234 - DNA, 44 bp, Artificial Sequence
  Synthetic oligonucleotide Blocker BC 91
atcgcatccttccgctgagtcggagacacgcagggatgagatgg

SEQ ID NO: 235 - DNA, 44 bp, Artificial Sequence
  Synthetic oligonucleotide Blocker BC 92
atcgcggttcctagctgagtcggagacacgcagggatgagatgg

SEQ ID NO: 236 - DNA, 44 bp, Artificial Sequence
  Synthetic oligonucleotide Blocker BC 93
atcgattggacaagctgagtcggagacacgcagggatgagatgg

SEQ ID NO: 237 - DNA, 43 bp, Artificial Sequence
  Synthetic oligonucleotide Blocker BC 94
atcgcttgtcggactgagtcggagacacgcagggatgagatgg

SEQ ID NO: 238 - DNA, 43 bp, Artificial Sequence
  Synthetic oligonucleotide Blocker BC 95
atcgatctgtccgctgagtcggagacacgcagggatgagatgg

SEQ ID NO: 239 - DNA, 43 bp, Artificial Sequence
  Synthetic oligonucleotide Blocker BC 96
atcgaccgcttaactgagtcggagacacgcagggatgagatgg

(19) United States
(12) Patent Application Publication
(10) Pub. No.: US 2014/0243232 A1
(43) Pub. Date: Aug. 28, 2014

(54) NUCLEIC ACID COMPLEXITY REDUCTION

(71) Applicants: Gavin Meredith, Cardiff, CA (US); Christopher Clouser, Salem, MA (US); Tanya Sokolsky, Cambridge, MA (US); Kimberly Mather, Williamstown, NJ (US); Kevin McKernan, Marblehead, MA (US); Marie Callahan, San Diego, CA (US); Gary Bee, Vista, CA (US)

(72) Inventors: Gavin Meredith, Cardiff, CA (US); Christopher Clouser, Salem, MA (US); Tanya Sokolsky, Cambridge, MA (US); Kimberly Mather, Williamstown, NJ (US); Kevin McKernan, Marblehead, MA (US); Marie Callahan, San Diego, CA (US); Gary Bee, Vista, CA (US)

(73) Assignee: LIFE TECHNOLOGIES CORPORATION, Carlsbad, CA (US)

(21) Appl. No.: 14/232,572

(22) PCT Filed: Jul. 13, 2012
(86) PCT No.: PCT/US2012/046624

§ 371 Date: Mar. 13, 2014

Related U.S. Application Data
(60) Provisional application No. 61/507,961, filed on Jul. 14, 2011.
(60) Provisional application No. 61/524,031, filed on Aug. 16, 2011.

(51) Int. Cl. C12Q 1/68; C07H 21/00

(52) U.S. Cl. CPC C12Q 1/6874; C07H 21/00
USPC 506/9; 536/25.41; 536/23.1; 435/6.11

(57) ABSTRACT

In some embodiments, the present teachings provide compositions, systems, methods and kits for reducing the complexity of nucleotide sequences in a nucleic acid sample comprising the steps: hybridizing a plurality of polynucleotide constructs to at least one blocker oligonucleotide and to at least one capture oligonucleotide, wherein the plurality of polynucleotide constructs include a plurality of polynucleotides each joined to at least one nucleic acid adaptor, wherein the at least one nucleic acid adaptor can hybridize to the at least one blocker oligonucleotide, and wherein the at least one capture oligonucleotide can hybridize to at least a portion of target polynucleotides that are a sub-population of the plurality of polynucleotides, so as to produce a capture duplex.

This application claims the filing date benefit of U.S. Provisional Application Nos. 61/507,961 filed on Jul. 14, 2011, 61/524,031 filed on Aug. 16, 2011, 61/529,687 filed on Aug. 31, 2011, and 61/545,290 filed on Oct. 10, 2011, the disclosures of which are incorporated herein by reference in their entireties.

Throughout this application various publications, patents, and/or patent applications are referenced. The disclosures of these publications, patents, and/or patent applications in their entireties are hereby incorporated by reference into this application in order to more fully describe the state of the art to which this invention pertains.

### FIELD

In some embodiments, the present teachings provide compositions, systems, methods and kits for reducing the complexity of nucleotide sequences in a nucleic acid sample comprising hybridizing a nucleic acid sample with one or more blocker oligonucleotides, and optionally with one or more nucleic acid capture oligonucleotides, to form a capture duplex.

### INTRODUCTION

Reducing the complexity of a nucleic acid sample produces a sub-population that is enriched for nucleic acids having desired sequences or lacking nucleic acids having undesirable sequences. Complexity reducing methods typically employ sequence-specific hybridization to capture nucleic acids from a source such as genomic DNA, DNA libraries or RNA. The resulting enriched population can be used as probes, or can be quantitated or sequenced.

### SUMMARY

In some embodiments, the present teachings provide compositions, systems, methods and kits for reducing the complexity of nucleotide sequences in a nucleic acid sample.

In some embodiments, methods for reducing the complexity of nucleotide sequences in a nucleic acid sample comprise capturing target polynucleotides from an initial nucleic acid sample to obtain a nucleic acid subpopulation having desired sequences or lacking certain sequences. In some embodiments, an initial nucleic acid sample includes target polynucleotides and non-target polynucleotides. In some embodiments, each of the target polynucleotides and non-target polynucleotides can be joined to at least one nucleic acid adaptor to form target and non-target polynucleotide constructs, respectively. In some embodiments, methods for capturing target polynucleotides comprise contacting an initial nucleic acid sample with an oligonucleotide under conditions suitable to hybridize the oligonucleotide to a nucleic acid adaptor. In some embodiments, the oligonucleotide can be a blocker oligonucleotide which hybridizes to the nucleic acid adaptor, thereby reducing or blocking non-specific hybridization between a nucleic acid adaptor and other nucleic acids in the hybridization reaction. Optionally, the methods further comprise contacting the initial nucleic acid sample with a capture oligonucleotide. In some embodiments, a capture oligonucleotide hybridizes to at least a portion of the target polynucleotide. In some embodiments, the capture oligonucleotide hybridizes to at least a portion of the target polynucleotide to form a capture duplex. Optionally, the capture duplexes can be separated from the non-target polynucleotides that do not form duplexes to enrich target polynucleotides. Optionally, the methods can further comprise sequencing a target polynucleotide that formed a capture duplex.

In some embodiments, methods for capturing target polynucleotides comprises: (a) providing a nucleic acid sample having a plurality of non-target polynucleotide constructs which include a plurality of non-target polynucleotides each joined to at least one nucleic acid adaptor, and the nucleic acid sample having a plurality of target polynucleotide constructs which include a plurality of target polynucleotides each joined to at least one nucleic acid adaptor; (b) contacting the nucleic acid sample with at least one blocker oligonucleotide which hybridizes with the at least one nucleic acid adaptor. Optionally, the method further comprises contacting the nucleic acid sample with at least one capture oligonucleotide which hybridizes to at least a portion of the plurality of target polynucleotides to form at least one capture duplex. Optionally, the methods further comprises separating the at least one capture duplex from non-target polynucleotides that do not form capture duplexes to enrich target polynucleotides. Optionally, the methods further comprise sequencing a target polynucleotide that formed a capture duplex.

In some embodiments, methods for capturing target polynucleotides comprises: (a) providing a nucleic acid sample having a plurality of single-stranded non-target polynucleotide constructs which include a plurality of non-target polynucleotides each joined to at least one nucleic acid adaptor, and the nucleic acid sample having a plurality of single-stranded target polynucleotide constructs which include a plurality of target polynucleotides each joined to at least one nucleic acid adaptor; (b) contacting the nucleic acid sample with at least one blocker oligonucleotide which hybridizes with the at least one nucleic acid adaptor. Optionally, the methods further comprise contacting the nucleic acid sample with at least one capture oligonucleotide which hybridizes to at least a portion of the plurality of single-stranded target polynucleotides to form at least one capture duplex. Optionally the methods further comprises: separating the at least one capture duplex from non-target polynucleotides that do not form capture duplexes to enrich target polynucleotides. Optionally, the methods further comprise sequencing a target polynucleotide that formed a capture duplex.

In some embodiments, methods for capturing target polynucleotides comprises: (a) providing a nucleic acid sample having a plurality of single-stranded non-target polynucleotide constructs which include a plurality of non-target polynucleotides each joined to a first and a second nucleic acid adaptor, and the nucleic acid sample having a plurality of single-stranded target polynucleotide constructs which include a plurality of target polynucleotides each joined to a first and a second nucleic acid adaptor; (b) contacting the nucleic acid sample with a first blocker oligonucleotide which hybridizes with the first nucleic acid adaptor; (c) contacting the nucleic acid sample with a second blocker oligonucleotide which hybridizes with the second nucleic acid adaptor. Optionally, the method further comprises contacting the nucleic acid sample with at least one capture oligonucleotide which hybridizes to at least a portion of the plurality of single-stranded target polynucleotides to form at least one capture duplex. Optionally the methods further comprises: separating the at least one capture duplex from non-target polynucleotides that do not form capture duplexes to enrich target polynucleotides. Optionally, the methods further comprise sequencing a target polynucleotide that formed a capture duplex.

In some embodiments, methods for capturing target polynucleotides comprises: (a) providing a nucleic acid sample having a plurality of double-stranded non-target polynucleotide constructs which include a plurality of non-target polynucleotides each joined to at least one nucleic acid adaptor, and the nucleic acid sample having a plurality of double-stranded target polynucleotide constructs which include a plurality of target polynucleotides each joined to at least one nucleic acid adaptor; (b) denaturing the nucleic acid sample to generate a single-stranded nucleic acid sample having a plurality of single-stranded non-target polynucleotide constructs and a plurality of single-stranded target polynucleotide constructs; (c) hybridizing the single-stranded nucleic acid sample to at least one blocker oligonucleotide which hybridizes to the at least one nucleic acid adaptor. Optionally, the method further comprises contacting the single-stranded nucleic acid sample with at least one capture oligonucleotide which hybridizes to at least a portion of the single-stranded target polynucleotide to produce a plurality of capture duplexes. Optionally the methods further comprises: separating the at least one capture duplex from non-target polynucleotides that do not form capture duplexes to enrich target polynucleotides. Optionally, the methods further comprise sequencing a target polynucleotide that formed a capture duplex.

In some embodiments, methods for capturing target polynucleotides comprises: (a) providing a nucleic acid sample having a plurality of double-stranded non-target polynucleotide constructs which include a plurality of non-target polynucleotides each joined to a first and a second nucleic acid adaptor, and the nucleic acid sample having a plurality of double-stranded target polynucleotide constructs which include a plurality of target polynucleotides each joined to a first and a second nucleic acid adaptor; (b) denaturing the nucleic acid sample to generate a single-stranded nucleic acid sample having a plurality of single-stranded non-target polynucleotide constructs and a plurality of single-stranded target polynucleotide constructs; (c) contacting the single-stranded nucleic acid sample with a first blocker oligonucleotide which hybridizes with the first nucleic acid adaptor; (d) contacting the single-stranded nucleic acid sample with a second blocker oligonucleotide which hybridizes with the second nucleic acid adaptor. Optionally, the method further comprises contacting the single-stranded nucleic acid sample with at least one capture oligonucleotide which hybridizes to at least a portion of the plurality of single-stranded target polynucleotides to form at least one capture duplex. Optionally the methods further comprises: separating the at least one capture duplex from non-target polynucleotides that do not form capture duplexes to enrich target polynucleotides. Optionally, the methods further comprise sequencing a target polynucleotide that formed a capture duplex.

The present teachings provide a capture duplex produced by any method disclosed herein.

Optionally, the methods further comprise separating at least one capture duplex from a plurality of non-target polynucleotide constructs.

In some embodiments, the separated capture duplex can form an enriched target polynucleotide.

The present teachings provide an enriched target polynucleotides produced by any method disclosed herein.

In some embodiments, the capture oligonucleotide comprises a binding moiety.

In some embodiments, the binding moiety comprises biotin.

Optionally, the method further comprises contacting the binding moiety to a binding partner moiety.

In some embodiments, the binding partner moiety comprises avidin or streptavidin.

In some embodiments, the binding partner moiety can be attached to a bead.

In some embodiments, the bead can be magnetic or paramagnetic.

In some embodiments, in the capture duplex, the capture oligonucleotide includes a binding moiety which binds a binding partner moiety (which is attached to a bead).

Optionally, the methods further comprise removing the bead from non-target polynucleotides.

In some embodiments, the bead can be removed from the non-target polynucleotides by contacting the bead (e.g., paramagnetic bead) to a magnetic source and separating the magnet-bead complex from the non-target polynucleotides to form separated capture duplexes.

In some embodiments, the separated capture duplexes form a plurality of enriched target polynucleotides.

The present teachings provide an enriched target polynucleotide produced by any method disclose herein.

In some embodiments, a target polynucleotide can be sequenced by any sequencing method, including sequencing-by-synthesis, ion-based sequencing involving the detection of sequencing byproducts using ISFETs, chemical degradation sequencing, ligation-based sequencing, hybridization sequencing, pyrophosphate detection sequencing, capillary electrophoresis, gel electrophoresis, next-generation, massively parallel sequencing platforms, sequencing platforms that detect hydrogen ions or other sequencing by-products, and single molecule sequencing platforms.

In some embodiments, a nucleic acid adaptor (e.g., a first nucleic acid adaptor) comprises a P1 adaptor sequence according to any one of SEQ ID NOS:3 or 5.

In some embodiments, a nucleic acid adaptor (e.g., a second nucleic acid adaptor) comprises an A adaptor sequence according to any one of SEQ ID NOS:140 or 141.

In some embodiments, a blocker oligonucleotide (e.g., a first blocker oligonucleotide) comprises a sequence according to SEQ ID NO:112 or 143.

In some embodiments, a blocker oligonucleotide (e.g., a second blocker oligonucleotide) comprises a sequence according to SEQ ID NOS:139 or 142.

In some embodiments, a barcoded blocker oligonucleotide (e.g., a second blocker oligonucleotide) comprises a sequence according to any one of SEQ ID NOS:144-239.

### DRAWINGS

FIG. 1 is a schematic depicting non-limiting embodiments of complexity reducing methods, and blocker oligonucleotides and capture oligonucleotides.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

### DEFINITIONS

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with exemplary embodiments provided herein, the following terms, unless otherwise indicated, shall be understood to have the following meanings:

As used herein the terms “hybridize” and “hybridization” and “hybridizing” (and other related terms) can include hydrogen bonding between two different nucleic acids, or between two different regions of a nucleic acid, to form a duplex nucleic acid. Hybridization can comprise Watson-Crick or Hoogstein binding to form a duplex nucleic acid. The two different nucleic acids, or the two different regions of a nucleic acid, may be complementary, or partially complementary. The complementary base pairing can be the standard A-T or C-G base pairing, or can be other forms of base-pairing interactions. Duplex nucleic acids can include mismatched nucleotides. Complementary nucleic acid strands need not hybridize with each other across their entire length.

### DESCRIPTION OF VARIOUS EMBODIMENTS

In some embodiments, the present teachings provide compositions, systems, methods and kits for reducing the complexity of nucleotide sequences in a nucleic acid sample.

The phrases “reducing the complexity of nucleotide sequences” and “reducing the complexity” and “reducing nucleic acid complexity” and “complexity reducing” and “reduced complexity nucleotide sequences” may be used interchangeably, and refer to capturing target polynucleotides from an initial nucleic acid sample to obtain a nucleic acid subpopulation having desired sequences or lacking certain sequences. In some embodiments, an initial nucleic acid sample includes non-target polynucleotide constructs and target polynucleotide constructs, where the target polynucleotide constructs can be selectively captured.

In some embodiments, methods for capturing target polynucleotides comprise increasing the efficiency of sequence-specific capture using oligonucleotides that block non-specific hybridization between the capture oligonucleotides and adaptor sequences in an initial nucleic acid sample. For example, blocker oligonucleotides can hybridize to at least a portion of a nucleic acid adaptor to decrease non-specific hybridization. In some embodiments, non-specific hybridization includes, renaturation of double-stranded polynucleotide constructs, hybridization between different polynucleotide constructs, secondary structure formation (e.g., stem-loops), or hybridization between a capture oligonucleotide and a nucleic acid adaptor sequence.

In some embodiments, capturing target polynucleotides comprises sequence-specific capture of polynucleotides of interest from a nucleic acid sample having a plurality of target and non-target polynucleotides. In some embodiments, capturing target polynucleotides comprises hybridizing polynucleotides of interest with one or more capture oligonucleotides. In some embodiments, a nucleic acid sample can include a plurality of polynucleotide constructs (both target and non-target) each having a polynucleotide joined to one or more adaptor sequences.

### Complexity Reducing Methods:

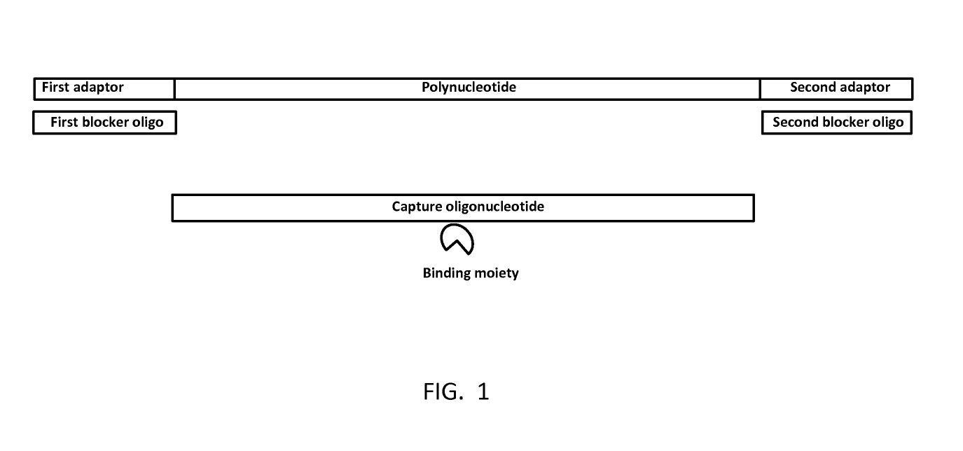

In some embodiments, methods for capturing target polynucleotides comprise contacting an initial nucleic acid sample with one or more blocker oligonucleotides (FIG. 1). In some embodiments, an initial nucleic acid sample includes target polynucleotides and non-target polynucleotides. In some embodiments, each of the target polynucleotides and the non-target polynucleotides can be joined to at least one nucleic acid adaptor to form target and non-target polynucleotide constructs, respectively. In some embodiments, methods for capturing target polynucleotides comprise contacting an initial nucleic acid sample with at least one blocker oligonucleotide under conditions suitable to hybridize the blocker oligonucleotide to a nucleic acid adaptor (FIG. 1). Optionally, the methods further comprise contacting the initial nucleic acid sample with a capture oligonucleotide (FIG. 1). In some embodiments, a blocker oligonucleotide hybridizes to at least a portion of the nucleic acid adaptor. In some embodiments, a capture oligonucleotide hybridizes to at least a portion of the target polynucleotide. In some embodiments, the capture oligonucleotide hybridizes to at least a portion of the target polynucleotide to form a capture duplex.

In some embodiments, methods for capturing target polynucleotides can further comprise separating the capture duplexes from nucleic acids in the initial sample that are not part of a capture duplex to enrich a target polynucleotide. In some embodiments, the capture duplexes can be denatured to release single-stranded target polynucleotides. In some embodiments, the capture duplexes can be nucleotide sequences having reduced complexity. In some embodiments, the enriched target polynucleotides can be nucleotide sequences having reduced complexity. In some embodiments, the released single-stranded target polynucleotides can be nucleotide sequences having reduced complexity.

In some embodiments, methods for capturing target polynucleotides comprise: (a) providing a nucleic acid sample having a plurality of non-target polynucleotide constructs which include a plurality of non-target polynucleotides each joined to at least one nucleic acid adaptor, and the nucleic acid sample having a plurality of target polynucleotide constructs which include a plurality of target polynucleotides each joined to at least one nucleic acid adaptor; (b) preventing non-specific hybridization by contacting the nucleic acid sample with at least one blocker oligonucleotide which hybridizes with the at least one nucleic acid adaptor. In some embodiments, the method further comprises contacting the nucleic acid sample with at least one capture oligonucleotide which hybridizes to at least a portion of the plurality of target polynucleotides to form at least one capture duplex. In some embodiments, the methods further comprises separating the at least one capture duplex from non-target polynucleotides that do not form capture duplexes to enrich target polynucleotides. In some embodiments, the methods further comprise sequencing a target polynucleotide that formed a capture duplex.

In some embodiments, methods for capturing target polynucleotides comprise: (a) providing a nucleic acid sample having a plurality of single-stranded polynucleotides each joined to at least a first adaptor sequence, wherein the plurality of single-stranded polynucleotides includes non-target polynucleotide sequences and at least one target polynucleotide sequence; (b) hybridizing a plurality of a first blocker oligonucleotide to the nucleic acid sample under suitable hybridization conditions, wherein the first blocker oligonucleotide includes a nucleotide sequence that hybridizes to at least a portion of the first adaptor sequence; (c) hybridizing a plurality of a capture oligonucleotide to the nucleic acid sample under suitable hybridization conditions to produce a plurality of capture duplexes, wherein the capture oligonucleotides include a sequence that hybridizes to at least a portion of the target polynucleotide sequence. In some embodiments, the capture oligonucleotide includes a binding moiety. In some embodiments, the methods further comprise: (d) separating the plurality of capture duplexes from the non-duplexed single-stranded polynucleotides by binding the binding moiety on the capture duplexes with a binding partner moiety. In some embodiments, the binding moiety comprises biotin. In some embodiments, the binding partner moiety comprises avidin or streptavidin.

In some embodiments, methods for capturing target polynucleotides comprise: (a) providing a nucleic acid sample having a plurality of single-stranded polynucleotides each joined to a first adaptor sequence and a second adaptor sequence, wherein the plurality of single-stranded polynucleotides includes non-target polynucleotide sequences at least one target polynucleotide sequence; (b) hybridizing a plurality of a first blocker oligonucleotide to the nucleic acid sample under suitable hybridization conditions, wherein the first blocker oligonucleotide includes a nucleotide sequence that hybridizes to the first adaptor sequence; (b) hybridizing a plurality of a second blocker oligonucleotide to the nucleic acid sample under suitable hybridization conditions, wherein the second blocker oligonucleotide includes a nucleotide sequence that hybridizes to the second adaptor sequence; (c) hybridizing a plurality of a capture oligonucleotide to the nucleic acid sample under suitable hybridization conditions to produce a plurality of capture duplexes, wherein the capture oligonucleotides include a sequence that hybridizes to at least a portion of the target polynucleotide sequence. In some embodiments, the capture oligonucleotide includes a binding moiety. In some embodiments, the methods further comprise: (d) separating the plurality of capture duplexes from the non-duplexed single-stranded polynucleotides by binding the binding moiety on the capture duplexes with a binding partner moiety. In some embodiments, the binding moiety comprises biotin. In some embodiments, the binding partner moiety comprises avidin or streptavidin.

In some embodiments, methods for capturing target polynucleotides comprise: (a) providing a nucleic acid sample having a plurality of double-stranded polynucleotides each joined to at least one adaptor sequence, wherein the plurality of double-stranded polynucleotides includes non-target polynucleotide sequences and at least one target polynucleotide sequence; (b) denaturing the double-stranded polynucleotides to generate a plurality of single-stranded polynucleotides each joined to at least one adaptor sequence; (c) hybridizing a plurality of a blocker oligonucleotide to the plurality of single-stranded polynucleotides under suitable hybridization conditions, wherein the blocker oligonucleotide includes a nucleotide sequence that hybridizes to the adaptor sequence; (d) hybridizing a plurality of a capture oligonucleotide to the plurality of the single-stranded polynucleotides under suitable hybridization conditions to produce a plurality of capture duplexes, wherein the capture oligonucleotides include a sequence that hybridizes to at least a portion of the target polynucleotide sequence. In some embodiments, the capture oligonucleotide includes a binding moiety. In some embodiments, the methods further comprise: (d) separating the plurality of capture duplexes from the non-duplexed single-stranded polynucleotides by binding the binding moiety on the capture duplexes with a binding partner moiety. In some embodiments, the binding moiety comprises biotin. In some embodiments, the binding partner moiety comprises avidin or streptavidin.

In some embodiments, methods for capturing target polynucleotides comprise: (a) providing a nucleic acid sample having a plurality of double-stranded polynucleotides each joined to a first adaptor sequence and a second adaptor sequence, wherein the plurality of double-stranded polynucleotides includes non-target polynucleotide sequences at least one target polynucleotide sequence; (b) denaturing the double-stranded polynucleotides to generate a plurality of single-stranded polynucleotides each joined to a first adaptor sequence and a second adaptor sequence; (c) hybridizing a plurality of a first blocker oligonucleotide to the plurality of the single-stranded polynucleotides under suitable hybridization conditions, wherein the first blocker oligonucleotide includes a nucleotide sequence that hybridizes to the first adaptor sequence; (d) hybridizing a plurality of a second blocker oligonucleotide to the plurality of the single-stranded polynucleotides under suitable hybridization conditions, wherein the second blocker oligonucleotide includes a nucleotide sequence that hybridizes to the second adaptor sequence; (e) hybridizing a plurality of a capture oligonucleotide to the plurality of the single-stranded polynucleotides under suitable hybridization conditions to produce a plurality of capture duplexes, wherein the capture oligonucleotides include a sequence that hybridizes to at least a portion of the target polynucleotide sequence. In some embodiments, the capture oligonucleotide includes a binding moiety. In some embodiments, the methods further comprise: (f) separating the plurality of capture duplexes from the non-duplexed single-stranded polynucleotides by binding the binding moiety on the capture duplexes with a binding partner moiety. In some embodiments, the binding moiety comprises biotin. In some embodiments, the binding partner moiety comprises avidin or streptavidin.

In some embodiments, a nucleic acid sample can include a plurality of polynucleotides having the same or different sequences. In some embodiments, a nucleic acid sample can comprise a plurality of target polynucleotides and/or non-target polynucleotides that are each joined to at least one nucleic acid adaptor to form target and non-target polynucleotide constructs which are part of a nucleic acid library (e.g., fragment libraries, barcoded fragment libraries, mate pair libraries and/or barcoded mate pair libraries). In some embodiments, a plurality of target polynucleotides and/or non-target polynucleotides can be joined to nucleic acid adaptors having the same or different sequences.

In some embodiments, a nucleic acid sample can initially include double stranded nucleic acids that can be denatured to form a plurality of single stranded nucleic acids. The single stranded nucleic acids can hybridize with the blocker oligonucleotides and/or hybridize with the capture oligonucleotides to form capture duplexes. In some embodiments, a nucleic acid sample can initially include single stranded nucleic acids that can hybridize with the blocker oligonucleotides and/or hybridize with the capture oligonucleotides to form capture duplexes.

In some embodiments, hybridization reactions can be conducted in an aqueous or non-aqueous solution. In some embodiments, hybridization reactions can be conducted under stringent or less-than-stringent hybridization conditions. In some embodiments, a nucleic acid sample can be hybridized essentially simultaneously with a mixture or with the same type of capture oligonucleotides, and/or with a mixture or with the same type of blocker oligonucleotides, and/or with a mixture or with the same type of capture oligonucleotides and blocker oligonucleotides. In some embodiments, a nucleic acid sample can be hybridized serially with one or more types of capture oligonucleotides or with one or more types of blocker oligonucleotides. In some embodiments, a combination of essentially simultaneous and/or serial hybridization modes can be used to hybridize a nucleic acid sample with capture oligonucleotides and/or with blocker oligonucleotides. In some embodiments, a nucleic acid sample can be hybridized to blocker oligonucleotides and/or to capture oligonucleotides in one round or multiple rounds of hybridization reactions.

In some embodiments, double-stranded capture oligonucleotides and/or double-stranded blocker oligonucleotides can be denatured to become single-stranded for hybridization to the polynucleotide constructs. In some embodiments, capture oligonucleotides and/or blocker oligonucleotides can initially be single-stranded for hybridization to the polynucleotide constructs.

In some embodiments, all steps of a target polynucleotide capture method can be conducted in one reaction vessel (e.g., a well) or different steps can be conducted in different reaction vessels (e.g., wells).

In some embodiments, enriched target polynucleotides generated from separate capture reactions can be pooled together. In some embodiments, different initial nucleic acid samples can be pooled together and then subjected to capture reactions.

In some embodiments, methods for capturing target polynucleotides can be conducted on any polynucleotide construct which is prepared for sequencing on any type of sequencing platform, including sequencing by oligonucleotide probe ligation and detection (e.g., SOLiD™ from Life Technologies, WO 2006/084131), probe-anchor ligation sequencing (e.g., Complete Genomics™ or Polonator™), sequencing-by-synthesis (e.g., Genetic Analyzer and HiSeq™, from IIlumina), pyrophosphate sequencing (e.g., Genome Sequencer FLX from 454 Life Sciences), ion-sensitive sequencing (e.g., Personal Genome Machine (PGM™) and Ion Proton™ Sequencer, both from Ion Torrent Systems, Inc.), and single molecule sequencing platforms (e.g., HeliScope™ from Helicos™).

In some embodiments, the first adaptor sequence can include a P1 sequence according to any one of SEQ ID NOS:1-5. In some embodiments, the second adaptor sequence can include a P2 sequence according to any one of SEQ ID NOS:6-12 or an A sequence according to any one of SEQ ID NOS:131-134, 140 or 141. In some embodiments, the second adaptor sequence can include a barcoded sequence according to any one of SEQ ID NOS:16-111. In some embodiments, the first blocker oligonucleotide can include a sequence that can hybridize to a P1 adaptor. For example, a first blocker oligonucleotide can include a sequence according to SEQ ID NO:112. In some embodiments, the second blocker oligonucleotide can include a sequence that can hybridize to an Ion A adaptor, an internal adaptor sequence, barcoded sequence or P2 adaptor sequence. For example, a second blocker oligonucleotide can include a sequence according to any of SEQ ID NO:139 or SEQ ID NOS:113-128.

### Enriching:

In some embodiments, methods for capturing target polynucleotides can further comprise separating the capture duplexes from nucleic acids in the sample that are not part of a capture duplex to enrich target polynucleotides. In some embodiments, a separating step can produce enriched target polynucleotides. In some embodiments, a separating step can be conducted with a paramagnetic bead separation reaction. In some embodiments, a capture oligonucleotide can be attached to a binding moiety (e.g., biotin). In some embodiments, a bead can be attached to a binding partner moiety (e.g., avidin or streptavidin). In some embodiments, the binding moiety can bind the binding partner moiety. In some embodiments, a bead can be magnetic or paramagnetic. In some embodiments, a separating step can comprise binding a capture oligonucleotide (which is hybridized to a portion of a target polynucleotide construct and which is attached to a binding moiety) to a paramagnetic bead which is attached to a binding partner moiety to form a capture duplex-bead complex. In some embodiments, a paramagnetic bead which is attached to a binding partner moiety comprises a Dynabeads™ M-270 (from Invitrogen, Carlsbad, Calif.). In some embodiments, a separating step can further include removing the capture duplex-bead complex from non-target polynucleotide constructs that do not form a duplex. In some embodiments, the removing step can employ a magnetic source to attract the paramagnetic bead, to separate the capture duplex-bead complex from the non-duplexes, to produce enriched target polynucleotides. In some embodiments, additional steps can include a washing step, for example to remove: unhybridized blocker oligonucleotides, unhybridized capture oligonucleotides and/or unhybridized polynucleotides. In some embodiments, enriched target polynucleotides can be nucleotide sequences having reduced complexity.

### Releasing:

In some embodiments, methods for capturing target polynucleotides can further comprise releasing enriched target polynucleotides. In some embodiments, releasing enriched target polynucleotides can include denaturing a capture duplex to release the target polynucleotide from the capture oligonucleotide, thereby producing a released target polynucleotide. For example, denaturing can include methods well known in the art for nucleic acid melting, such as employing any combination of elevated temperatures, decreased salt concentrations (e.g., sodium), and/or increased formamide concentrations. Guidance for calculating an appropriate melting conditions can be found in Casey and Davidson 1977 Nucleic Acids Research 4:1539; Breslauer et al., 1986 Proceedings National Academy of Science USA 83(11):3746-3750; and Rychlik et al., 1990 Nucleic Acids Research 18(21):6409-6412. Typically, nucleic acid melting conditions employ temperatures above the melting temperature for a given nucleic acid length and % GC content. In some embodiments, released target polynucleotides can be nucleotide sequences having reduced complexity. In some embodiments, target polynucleotides are not released from a capture duplex. In some embodiments, target polynucleotides remain hybridized to at least one capture oligonucleotide (bound or unbound to a bead).

### Hybridizing and Washing Conditions

In some embodiments, in the methods for capturing target polynucleotides, conditions that are suitable for nucleic acid hybridization and/or washing conditions include parameters such as salts, buffers, pH, temperature, GC % content of the capture oligonucleotides, GC % content of the blocker oligonucleotides, GC % content of the target polynucleotide, and/or time. For example, conditions suitable for hybridizing or washing polynucleotides with capture oligonucleotides and/or with blocker oligonucleotides can include hybridization solutions having sodium salts, such as NaCl, sodium citrate and/or sodium phosphate. In some embodiments, hybridization or wash solutions can include about 10-75% formamide and/or about 0.01-0.7% sodium dodecyl sulfate (SDS). In some embodiments, a hybridization solution can be a stringent hybridization solution which can include any combination of 50% formamide, 5×SSC (0.75 M NaCl, 0.075 M sodium citrate), 50 mM sodium phosphate (pH 6.8), 0.1% sodium pyrophosphate, 5×Denhardt's solution, 0.1% SDS, and/or 10% dextran sulfate. In some embodiments, the hybridization or washing solution can include any combination of non-specific competitor nucleic acids such as human Cot-1 DNA and/or salmon sperm DNA. In some embodiments, the hybridization or washing solution can include BSA (bovine serum albumin).

In some embodiments, hybridization or washing can be conducted at a temperature range of about 25-90° C., or about 30-75° C., or about 40-60° C., or about 60-80° C., or about 80-99° C., or higher.

In some embodiments, hybridization or washing can be conducted for a time range of about 1-6 hours, or about 6-12 hours, or about 12-24 hours, or about 24-36 hours, or about 36-48 hours, or about 2-3 days, or about 3-4 days, or about 4-5 days, or about 5-6 days, or about 6-7 days, or about 7-8 days, or more than 8 days.

In some embodiments, hybridization or wash conditions can be conducted at a pH range of about 5-10, or about pH 6-9, or about pH 6.5-8, or about pH 6.5-7.

In some embodiments, capture oligonucleotides and/or blocker oligonucleotides can be reacted with an amount of single-stranded or double-stranded polynucleotide constructs (e.g., nucleic acid library) of about 100-250 ng, or about 250-500 ng, or about 500-650 ng, or about 650-800 ng, or about 800-1000 ng, or more.

Methods for nucleic acid hybridization and washing are well known in the art. For example, thermal melting temperature (Tₘ) for nucleic acids can be a temperature at which half of the nucleic acid strands are double-stranded and half are single-stranded under a defined condition. In some embodiments, a defined condition can include ionic strength and pH in an aqueous reaction condition. A defined condition can be modulated by altering the concentration of salts (e.g., sodium), temperature, pH, buffers, and/or formamide. Typically, the calculated thermal melting temperature can be at about 5-30° C. below the Tₘ, or about 5-25° C. below the Tₘ, or about 5-20° C. below the Tₘ, or about 5-15° C. below the Tₘ, or about 5-10° C. below the Tₘ. Methods for calculating a Tₘ are well known and can be found in Sambrook (1989 in “Molecular Cloning: A Laboratory Manual”, 2^{nd} edition, volumes 1-3; Wetmur 1966, J. Mol. Biol., 31:349-370; Wetmur 1991 Critical Reviews in Biochemistry and Molecular Biology, 26:227-259). Other sources for calculating a Tₘ for hybridizing or denaturing nucleic acids include OligoAnalyze (from Integrated DNA Technologies) and Primer3 (distributed by the Whitehead Institute for Biomedical Research).

### Compositions

In some embodiments, the present teachings provide a blocker oligonucleotide comprising an oligonucleotide that can hybridize to at least a portion of a nucleic acid adaptor. In some embodiments, a blocker oligonucleotide can be a nucleic acid, including double-stranded, single-stranded, DNA, RNA or DNA/RNA hybrid. In some embodiments, a blocker oligonucleotide comprises a sequence having full or partial complementarity with a nucleic acid adaptor. In some embodiments, the sequence and length of a blocker oligonucleotide can be designed based on the sequence and length of any nucleic acid adaptor. For example, nucleic acid adaptors include sequences according to any of SEQ ID NOS: 1-12, 15-111, 129-138, and 140, 141. For example, blocker oligonucleotides include sequences according to any of SEQ ID NOS: 112-128, 139 and 142-239.

In some embodiments, the present teachings provide a capture duplex comprising a polynucleotide construct hybridized to at least one capture oligonucleotide. In some embodiments, a capture oligonucleotide can be a nucleic acid, including double-stranded, single-stranded, DNA, RNA or DNA/RNA hybrid. In some embodiments, a capture oligonucleotide comprises a sequence having full or partial complementarity with at least a portion of at least one target polynucleotide sequence. In some embodiments, the sequence and length of a capture oligonucleotide can be designed based on the sequence and length of any target polynucleotide sequence. In some embodiments, a capture duplex can also be hybridized to at least one blocker oligonucleotide. In some embodiments, the polynucleotide construct includes a polynucleotide joined to at least one nucleic acid adaptor. In some embodiments, the polynucleotide (which is joined to the at least one adaptor) can be a target polynucleotide. In some embodiments, the capture oligonucleotide can hybridize to at least a portion of the target polynucleotide. In some embodiments, the blocker oligonucleotide can hybridize to at least a portion of the nucleic acid adaptor. In some embodiments, a nucleic acid adaptor comprises a sequence according to any of SEQ ID NOS: 1-12, 15-111, 129-138, and 140, 141. In some embodiments, a blocker oligonucleotide comprises a sequence according to any of SEQ ID NOS: 112-128, 139 and 142-239. In some embodiments, a capture duplex can be located among a population of duplexed and non-duplexed polynucleotide constructs. In some embodiments, a capture duplex can be generated by employing any method described herein or methods well known in the art.

In some embodiments, the present teachings provide an enriched target polynucleotide comprising a capture duplex that is separated away from polynucleotide constructs that do not form duplexes. In some embodiments, a capture duplex comprises a polynucleotide construct hybridized to at least one capture oligonucleotide. In some embodiments, a capture duplex can also be hybridized to at least one blocker oligonucleotide. In some embodiments, an enriched target polynucleotide can be attained by conducting a paramagnetic bead separation reaction to separate a capture duplex from polynucleotide constructs that do not form duplexes with a capture oligonucleotide. In some embodiments, an enriched target polynucleotide can be washed to remove unhybridized blocker oligonucleotides, unhybridized capture oligonucleotides and/or unhybridized polynucleotides. In some embodiments, the capture oligonucleotide can be attached to a binding moiety. For example, the binding moiety can be biotin. In some embodiments, a bead can be attached to a binding partner moiety, wherein the binding partner moiety binds to a binding moiety. For example, the binding partner moiety can be avidin or streptavidin. In some embodiments, the bead can be magnetic or paramagnetic. In some embodiments, a population of capture duplexes and non-duplex polynucleotide constructs can be contacted with a bead which is attached to a binding partner moiety. In some embodiments, a capture duplex can be contacted with a bead which is attached to a binding partner moiety, wherein the binding partner moiety can bind the binding moiety on the capture polynucleotide (which is hybridized to the target polynucleotide). In some embodiments, a paramagnetic bead (which is attached to a binding partner moiety) can bind to a binding moiety. In some embodiments, an enriched target polynucleotide can be denatured to separate the target polynucleotide construct from the capture oligonucleotide, to produce a released target polynucleotide. In some embodiments, a denaturing step can be omitted so that a target polynucleotide construct remains hybridized to a capture oligonucleotide. In some embodiments, enriched target polynucleotides and released target polynucleotides can be generated by employing any method described herein or methods well known in the art.

### Blocker Oligonucleotides

In some embodiments, the present teachings provide blocker oligonucleotides comprising an oligonucleotide. In some embodiments, blocker oligonucleotides can be DNA, cDNA, RNA, RNA/DNA hybrids, or analogs thereof. Blocker oligonucleotides can be single-stranded or double-stranded nucleic acids (or analogs thereof). Blocker oligonucleotides can include one or more nucleotide or nucleoside analogs, such as locked nucleic acids (LNA). Blocker oligonucleotides can be any length, including about 5-10 bp, or about 10-20 bp, or about 20-30 bp, or about 30-40 bp, or about 40-50 bp, or about 50-60 bp, or about 60-70 bp, or about 70-80 bp, or about 80-90 bp, or about 90-100 bp, or longer.

In some embodiments, blocker oligonucleotides can include degenerate bases. In some embodiments, blocker oligonucleotides can include one or more inosine residues.

In some embodiments, blocker oligonucleotides can include at least one scissile linkage. In some embodiments, a scissile linkage can be susceptible to cleavage or degradation by an enzyme or chemical compound. In some embodiments, blocker oligonucleotides can include at least one phosphorothiolate, phosphorothioate, and/or phosphoramidate linkage.

In some embodiments, blocker oligonucleotides can include nucleotide sequences that can hybridize to any portion of a polynucleotide construct. For example, a blocker oligonucleotide can hybridize to an adaptor sequence. In some embodiments, blocker oligonucleotides can include nucleotide sequences that are fully complementary (e.g., base pairing A-T and/or C/G) or partially complementary (e.g., mis-match pairing A with C or G, T with C or G, C with A or T, or G with A or T) to any portion of the polynucleotides or polynucleotide constructs. In some embodiments, blocker oligonucleotides can include nucleotide sequences that are complementary to at least a portion of one or more adaptors (e.g., P1, P2, A, internal, barcoded or universal adaptors) or can include nucleotide sequences that are complementary to a sequencing primer or amplification primer sequence, for example a primer sequence selected from SEQ ID NOS:112-128). In some embodiments, blocker oligonucleotides can include nucleotide sequences of at least a portion of one or more adaptors (e.g., P1, P2, A, internal, barcoded or universal adaptors) or a sequencing primer or amplification primer sequence.

In some embodiments, blocker oligonucleotides can include nucleotide sequences selected from SEQ ID NOS:112-128, 135-139 and 142-239. These can optionally be used in conjunction with adapters and/or primers including nucleotide sequences selected from SEQ ID NOS: 129-134.

In some embodiments, blocker oligonucleotides can include nucleotide sequences that are complementary to any combination of one or more adaptors. For example, blocker oligonucleotides can include nucleotide sequences that are complementary to any one or any combination of adaptor sequences, including: P1; P2; A; internal adaptor; and/or any barcode adaptor (any of SEQ ID NOS:16-111). One skilled in the art will recognize that many combinations are possible.

Blocker oligonucleotides can include nucleotide sequence that can hybridize to any adaptor sequence that can be used to construct any type of nucleic acid library, including adaptor sequences for: SOLiD™ library (from Life Technologies, WO 2006/084131), Complete Genomics™ library, Polonator™ library, Genetic Analyzer library (Illumina), HiSeq™ library (Illumina), Genome Sequencer FLX library (454 Life Sciences), Personal Genome Machine library (Ion Torrent Systems, Inc.), Ion Proton™ Sequencer (Ion Torrent Systems, Inc.) and HeliScope™ library (Helicos™).

### Polynucleotides:

In some embodiments, the present teachings provide compositions and methods for capturing target polynucleotides, where polynucleotides can be DNA, RNA, chimeric RNA/DNA, or analogs thereof. In some embodiments, polynucleotides can be single-stranded or double-stranded nucleic acids. In some embodiments, polynucleotides can be isolated in any form including chromosomal, genomic, organellar (e.g., mitochondrial, chloroplast or ribosomal), recombinant molecules, cloned, amplified (e.g., PCR amplified), cDNA, RNA (e.g., precursor mRNA, mRNA, miRNA, miRNA binding sites, fRNA), oligonucleotide, or any type of nucleic acid library. In some embodiments, polynucleotides can be isolated from any source including from organisms such as prokaryotes, eukaryotes (e.g., humans, plants and animals), fungus, and viruses; cells; tissues; normal or diseased cells or tissues or organs, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods. In some embodiments, polynucleotides can be chemically synthesized to include any type of nucleic acid analog. In some embodiments, polynucleotides can be isolated from a formalin-fixed tissue, or from a paraffin-embedded tissue, or from a formalin-fix paraffin-embedded (FFPE) tissue.

In some embodiments, polynucleotides can be polynucleotide fragments which can be generated enzymatically, chemically, or using any type of physical force (e.g., sonication, nebulization, or cavitation). For example, polynucleotides can be enzymatically fragmented by reacting with a restriction endonuclease. In another example, polynucleotides can be enzymatically fragmented by nicking and nick translating the nick (in the presence or absence of nucleic acid binding proteins) to generate double-stranded breaks using any method disclosed in application No. PCT/US2012/039691, filed May 25, 2012, or U.S. Ser. No. 13/482,542, filed May 29, 2012. In yet another example, polynucleotides can be enzymatically fragmented by binding polynucleotides with histones and cleaving with a nuclease (U.S. Pat. No. 8,202,691, issued Jun. 19, 2012). In some embodiments, polynucleotide fragments can be about 100-200 bp, or about 200-250 bp, or about 250-300 bp, or about 300-400 bp, or about 400-500 bp, or about 500-1000 bp, or about 100 bp-1000 bp, or about 1 kb-50 kb, or about 50 kb-100 kb, or about 100-250 kb, or about 250-500 kb, or about 500-750 kb, or about 750-1000 bp, or about 1000 bp to about 1 Mb, or about 1-10 Mb, or about 10-20 Mb, or about 20-30 Mb, or about 30-40 Mb, or about 40-50 Mb, or longer.

In some embodiments, methods for capturing target polynucleotides can be conducted with starting nucleic acid fragments in an amount of about 0.01-0.1 ng, or about 0.1-1 ng, or about 1-5 ng, or about 5-10 ng, or about 10-50 ng, or about 50-100 ng, or about 100-500 ng, or about 500-1000 ng, or about 1-2 ug, or about 2-5 ug, or about 5-10 ug, or about 10-50 ug, or about 50-100 ug, or about 100-500 ug, or about 500-1000 ug, or more.

### Polynucleotide Constructs

In some embodiments, the present teachings provide compositions and methods for capturing target polynucleotides, where at least one end of polynucleotides can be joined to any combination of one or more nucleic acid adaptors to form a polynucleotide construct. In some embodiments, one or both ends of a polynucleotide can be joined to at least one nucleic acid adaptor to generate a polynucleotide construct (e.g., FIG. 1). In some embodiments, one end of a polynucleotide can be joined to a first adaptor and the other end of the polynucleotide can be joined to a second adaptor. In some embodiments, the first and second adaptors can be the same or different adaptors. Nucleic acid adaptors can include sequences: P1, P2, A, internal adaptor, barcoded sequences, amplification primer sequences, sequencing primer sequences, and complementary sequences thereof. For example, polynucleotides can be joined to a first adaptor (e.g., P1 adaptor) and a second adaptor (e.g., A, internal adaptor, barcode and/or P2 adaptors) (FIG. 1). In some embodiments, polynucleotides and adaptors can be joined by ligation. In some embodiments, a polynucleotide can be joined to an adaptor with a ligase enzyme. In some embodiments, a polynucleotide can be joined to an adaptor by annealing or by conducting a primer extension reaction. In some embodiments, the length of polynucleotide constructs (e.g., polynucleotide joined to at least one adaptor) can be about 100-200 bp, or about 200-250 bp, or about 250-300 bp, or about 300-400 bp, or about 400-500 bp, or about 500-1000 bp, or about 100 bp-1000 bp, or longer.

### Adaptors

In some embodiments, the present teachings provide compositions and methods for capturing target polynucleotides, where a polynucleotide can be joined to one or more nucleic acid adaptors. In some embodiments, a nucleic acid adaptor (e.g., a first and/or second adaptor) can be DNA, RNA, chimeric RNA/DNA molecules, or analogs thereof. In some embodiments, an adaptor can include one or more ribonucleoside residues. In some embodiments, an adaptor can be single-stranded or double-stranded nucleic acids, or can include single-stranded and/or double-stranded portions. In some embodiments, an adaptor can have any structure, including linear, hairpin, forked, or stem-loop.

In some embodiments, an adaptor can be a blocking oligonucleotide adaptor which comprises a double-stranded oligonucleotide adaptor (duplex) having an overhang cohesive portion. In some embodiments, the overhang cohesive portions of a pair of blocking oligonucleotide adaptors can hybridize with each other. In some embodiments, each end of a polynucleotide can be joined to a blocker oligonucleotide and the cohesive portions can be hybridized to each other to generate a circular nucleic acid molecule.

In some embodiments, an adaptor can have any length, including fewer than 10 bases in length, or about 10-20 bases in length, or about 20-50 bases in length, or about 50-100 bases in length, or longer.

In some embodiments, an adaptor can have any combination of blunt end(s) and/or sticky end(s). In some embodiments, at least one end of an adaptor can be compatible with at least one end of a nucleic acid fragment. In some embodiments, a compatible end of an adaptor can be joined to a compatible end of a nucleic acid fragment. In some embodiments, an adaptor can have a 5′ or 3′ overhang end.

In some embodiments, an adaptor can include a monomeric sequence (e.g., AAA, TTT, CCC, or GGG) of any length, or an adaptor can include a complex sequence (e.g., non-monomeric sequence), or can include both monomeric and complex sequences.

In some embodiments, an adaptor can have a 5′ or 3′ tail. In some embodiments, the tail can be one, two, three, or more nucleotides in length. In some embodiments, an adaptor can have a tail comprising A, T, C, G and/or U. In some embodiments, an adaptor can have a monomeric tail sequence of any length. In some embodiments, at least one end of an adaptor can have a tail that is compatible with a tail on one end of a nucleic acid fragment.

In some embodiments, an adaptor can include an internal nick. In some embodiments, an adaptor can have at least one strand that lacks a terminal 5′ phosphate residue. In some embodiments, an adaptor lacking a terminal 5′ phosphate residue can be joined to a nucleic acid fragment to introduce a nick at the junction between the adaptor and the nucleic acid fragment.

In some embodiments, an adaptor can include a nucleotide sequence that is part of, or is complementary to, a P1 sequence (SEQ ID NOS:1-5), P2 sequence (SEQ ID NOS:6-12), A adaptor sequence (SEQ ID NOS:140-141), internal adaptor, barcode sequence (SEQ ID NOS:16-111, 133-134), amplification sequence (SEQ ID NOS:13 or 14), or a sequencing primer sequence, or any portion thereof. In some embodiments, an adaptor can include degenerate sequences. In some embodiments, an adaptor can include one or more inosine residues. In some embodiments, a barcode adaptor can include a uniquely identifiable sequence. In some embodiments, a barcode adaptor can be used for constructing multiplex nucleic acid libraries.

In some embodiments, an adaptor can include at least one scissile linkage. In some embodiments, a scissile linkage can be susceptible to cleavage or degradation by an enzyme or chemical compound. In some embodiments, an adaptor can include at least one phosphorothiolate, phosphorothioate, and/or phosphoramidate linkage.

In some embodiments, an adaptor can include identification sequences. In some embodiments, an identification sequences can be used for sorting or tracking. In some embodiments, an identification sequences can be a unique sequence (e.g., barcode sequence). In some embodiments, a barcode sequence can allow identification of a particular adaptor among a mixture of different adaptors having different barcodes sequences. For example, a mixture can include 2, 3, 4, 5, 6, 7-10, 10-50, 50-100, 100-200, 200-500, 500-1000, or more different adaptors having unique barcode sequences.

In some embodiments, an adaptor can include any type of restriction enzyme recognition sequence, including type I, type II, type Hs, type IIB, type III or type IV restriction enzyme recognition sequences.

In some embodiments, an adaptor can include a cell regulation sequences, including a promoter (inducible or constitutive), enhancers, transcription or translation initiation sequence, transcription or translation termination sequence, secretion signals, Kozak sequence, cellular protein binding sequence, and the like.

TABLE I

| | | SEQ ID |
|---|---|---|
| Adaptors: | Sequence 5′>3′ | NO: |
| P1-Adaptor | CCACTACGCCTCCGCTTTCCTCTCTATGGGCAGTCGGTGFT | 1 |
| (top strand) | | |
| P1-Adaptor | TCACCGACTGCCCATAGAGAGGAAAGCGGAGGCGTAGTGEOC | 2 |
| (bottom strand) | | |
| P1-Adaptor | CCACTACGCCTCCGCTTTCCTCTCTATGGGCAGTCGGTGAT | 3 |
| (top strand) | | |
| P1-Adaptor | ATCACCGACTGCCCATAGAGAGGAAAGCGGAGGCGTAGTGGTT | 4 |
| (bottom strand) | | |
| P1-Adaptor | ATCACCGACTGCCCATAGAGAGGAAAGCGGAGGCGTAGTGGCC | 5 |
| (bottom strand) | | |
| P2-Adaptor | AGAGAATGAGGAACCCGGGGCAGTT | 6 |
| (top strand) | | |
| P2-Adaptor | CTGCCCCGGGTTCCTCATTCTCT | 7 |
| (bottom strand) | | |
| P2-Adaptor | AGAGAATGAGGAACCCGGGGCAGTT | 8 |
| (top strand) | | |
| P2-Adaptor | AGAGAATGAGGAACCCGGGGCAGCC | 9 |
| (top strand) | | |
| P2-Adaptor | CTGCCCCGGGTTCCTCATTCTCT | 10 |
| (bottom strand) | | |
| P2-Adaptor | GAGAATGAGGAACCCGGGGCAEOC | 11 |
| (top strand) | | |
| P2-Adaptor | CTGCCCCGGGTTCCTCATTCTOT | 12 |
| (bottom strand) | | |

LEGEND:
F = A-3′phosphorothioate
E = G-3′phosphorothioate
O = C-3′phosphorothioate

TABLE II

| | | SEQ ID |
|---|---|---|
| Primers: | Sequence 5′>3′ | NO: |
| PCR1 primer | CCACTACGCCTCCGCTTTCCTCTCTATG | 13 |
| PCR2 primer | CTGCCCCGGGTTCCTCATTCT | 14 |

TABLE III

| | | SEQ ID |
|---|---|---|
| Barcodes: | Sequence 5′>3′ | NO: |
| Universal | CGCCTTGGCCGTACAGCAG | 15 |
| adaptor | | |
| BC-001 | CTGCCCCGGGTTCCTCATTCTCZETGTAAGAGGCTGCTGTACGGCCAAGGCET | 16 |
| BC-002 | CTGCCCCGGGTTCCTCATTCTCZFGGGAGTGGTCTGCTGTACGGCCAAGGCET | 17 |
| BC-003 | CTGCCCCGGGTTCCTCATTCTCZFTAGGTTATACTGCTGTACGGCCAAGGCET | 18 |
| BC-004 | CTGCCCCGGGTTCCTCATTCTCZEGATGCGGTCCTGCTGTACGGCCAAGGCET | 19 |
| BC-005 | CTGCCCCGGGTTCCTCATTCTCZETGGTGTAAGCTGCTGTACGGCCAAGGCET | 20 |
| BC-006 | CTGCCCCGGGTTCCTCATTCTCZECGAGGGACACTGCTGTACGGCCAAGGCET | 21 |
| BC-007 | CTGCCCCGGGTTCCTCATTCTCZEGGTTATGCCCTGCTGTACGGCCAAGGCET | 22 |
| BC-008 | CTGCCCCGGGTTCCTCATTCTCZEAGCGAGGATCTGCTGTACGGCCAAGGCET | 23 |
| BC-009 | CTGCCCCGGGTTCCTCATTCTCZFGGTTGCGACCTGCTGTACGGCCAAGGCET | 24 |
| BC-010 | CTGCCCCGGGTTCCTCATTCTCZECGGTAAGCTCTGCTGTACGGCCAAGGCET | 25 |
| BC-011 | CTGCCCCGGGTTCCTCATTCTCZETGCGACACGCTGCTGTACGGCCAAGGCET | 26 |
| BC-012 | CTGCCCCGGGTTCCTCATTCTCZFAGAGGAAAACTGCTGTACGGCCAAGGCET | 27 |
| BC-013 | CTGCCCCGGGTTCCTCATTCTCZECGGTAAGGCCTGCTGTACGGCCAAGGCET | 28 |
| BC-014 | CTGCCCCGGGTTCCTCATTCTCZETGCGGCAGACTGCTGTACGGCCAAGGCET | 29 |
| BC-015 | CTGCCCCGGGTTCCTCATTCTCZEAGTTGAATGCTGCTGTACGGCCAAGGCET | 30 |
| BC-016 | CTGCCCCGGGTTCCTCATTCTCZEGGAGACGTTCTGCTGTACGGCCAAGGCET | 31 |
| BC-017 | CTGCCCCGGGTTCCTCATTCTCZEGCTCACCGCCTGCTGTACGGCCAAGGCET | 32 |
| BC-018 | CTGCCCCGGGTTCCTCATTCTCZFGGCGGATGACTGCTGTACGGCCAAGGCET | 33 |
| BC-019 | CTGCCCCGGGTTCCTCATTCTCZFTGGTAACTGCTGCTGTACGGCCAAGGCET | 34 |
| BC-020 | CTGCCCCGGGTTCCTCATTCTCZETCAAGCTTTCTGCTGTACGGCCAAGGCET | 35 |
| BC-021 | CTGCCCCGGGTTCCTCATTCTCZETGCGGTTCCCTGCTGTACGGCCAAGGCET | 36 |
| BC-022 | CTGCCCCGGGTTCCTCATTCTCZEAGAAGATGACTGCTGTACGGCCAAGGCET | 37 |
| BC-023 | CTGCCCCGGGTTCCTCATTCTCZECGGTGCTTGCTGCTGTACGGCCAAGGCET | 38 |
| BC-024 | CTGCCCCGGGTTCCTCATTCTCZEGGTCGGTATCTGCTGTACGGCCAAGGCET | 39 |
| BC-025 | CTGCCCCGGGTTCCTCATTCTCZFACATGATGACTGCTGTACGGCCAAGGCET | 40 |
| BC-026 | CTGCCCCGGGTTCCTCATTCTCZOGGGAGCCCGCTGCTGTACGGCCAAGGCET | 41 |
| BC-027 | CTGCCCCGGGTTCCTCATTCTCZOAGCAAACTTCTGCTGTACGGCCAAGGCET | 42 |
| BC-028 | CTGCCCCGGGTTCCTCATTCTCZFGCTTACTACCTGCTGTACGGCCAAGGCET | 43 |
| BC-029 | CTGCCCCGGGTTCCTCATTCTCZEAATCTAGGGCTGCTGTACGGCCAAGGCET | 44 |
| BC-030 | CTGCCCCGGGTTCCTCATTCTCZETAGCGAAGACTGCTGTACGGCCAAGGCET | 45 |
| BC-031 | CTGCCCCGGGTTCCTCATTCTCZECTGGTGCGTCTGCTGTACGGCCAAGGCET | 46 |
| BC-032 | CTGCCCCGGGTTCCTCATTCTCZEGTTGGGTGCCTGCTGTACGGCCAAGGCET | 47 |
| BC-033 | CTGCCCCGGGTTCCTCATTCTCZOGTTGGATACCTGCTGTACGGCCAAGGCET | 48 |
| BC-034 | CTGCCCCGGGTTCCTCATTCTCZZCGTTAAAGGCTGCTGTACGGCCAAGGCET | 49 |
| BC-035 | CTGCCCCGGGTTCCTCATTCTCZFAGCGTAGGACTGCTGTACGGCCAAGGCET | 50 |
| BC-036 | CTGCCCCGGGTTCCTCATTCTCZETTCTCACATCTGCTGTACGGCCAAGGCET | 51 |
| BC-037 | CTGCCCCGGGTTCCTCATTCTCZOTGTTATACCCTGCTGTACGGCCAAGGCET | 52 |
| BC-038 | CTGCCCCGGGTTCCTCATTCTCZETCGTCTTAGCTGCTGTACGGCCAAGGCET | 53 |
| BC-039 | CTGCCCCGGGTTCCTCATTCTCZZATCGTGAGTCTGCTGTACGGCCAAGGCET | 54 |
| BC-040 | CTGCCCCGGGTTCCTCATTCTCZFAAAGGGTTACTGCTGTACGGCCAAGGCET | 55 |
| BC-041 | CTGCCCCGGGTTCCTCATTCTCZZGTGGGATTGCTGCTGTACGGCCAAGGCET | 56 |
| BC-042 | CTGCCCCGGGTTCCTCATTCTCZEAATGTACTACTGCTGTACGGCCAAGGCET | 57 |
| BC-043 | CTGCCCCGGGTTCCTCATTCTCZOGCTAGGGTTCTGCTGTACGGCCAAGGCET | 58 |
| BC-044 | CTGCCCCGGGTTCCTCATTCTCZFAGGATGATCCTGCTGTACGGCCAAGGCET | 59 |
| BC-045 | CTGCCCCGGGTTCCTCATTCTCZETACTTGGCTCTGCTGTACGGCCAAGGCET | 60 |
| BC-046 | CTGCCCCGGGTTCCTCATTCTCZEGTCGTCGAACTGCTGTACGGCCAAGGCET | 61 |
| BC-047 | CTGCCCCGGGTTCCTCATTCTCZEAGGGATGGCCTGCTGTACGGCCAAGGCET | 62 |
| BC-048 | CTGCCCCGGGTTCCTCATTCTCZECCGTAAGTGCTGCTGTACGGCCAAGGCET | 63 |
| BC-049 | CTGCCCCGGGTTCCTCATTCTCZFTGTCATAAGCTGCTGTACGGCCAAGGCET | 64 |
| BC-050 | CTGCCCCGGGTTCCTCATTCTCZEAAGGCTTGCCTGCTGTACGGCCAAGGCET | 65 |
| BC-051 | CTGCCCCGGGTTCCTCATTCTCZFAGCAGGAGTCTGCTGTACGGCCAAGGCET | 66 |
| BC-052 | CTGCCCCGGGTTCCTCATTCTCZETAATTGTAACTGCTGTACGGCCAAGGCET | 67 |
| BC-053 | CTGCCCCGGGTTCCTCATTCTCZETCATCAAGTCTGCTGTACGGCCAAGGCET | 68 |
| BC-054 | CTGCCCCGGGTTCCTCATTCTCZFAAAGGCGGACTGCTGTACGGCCAAGGCET | 69 |
| BC-055 | CTGCCCCGGGTTCCTCATTCTCZFGCTTAAGCGCTGCTGTACGGCCAAGGCET | 70 |
| BC-056 | CTGCCCCGGGTTCCTCATTCTCZECATGTCACCCTGCTGTACGGCCAAGGCET | 71 |
| BC-057 | CTGCCCCGGGTTCCTCATTCTCZOTAGTAAGAACTGCTGTACGGCCAAGGCET | 72 |
| BC-058 | CTGCCCCGGGTTCCTCATTCTCZZAAAGTGGCGCTGCTGTACGGCCAAGGCET | 73 |
| BC-059 | CTGCCCCGGGTTCCTCATTCTCZFAGTAATGTCCTGCTGTACGGCCAAGGCET | 74 |
| BC-060 | CTGCCCCGGGTTCCTCATTCTCZETGCCTCGGTCTGCTGTACGGCCAAGGCET | 75 |
| BC-061 | CTGCCCCGGGTTCCTCATTCTCZFAGATTATCGCTGCTGTACGGCCAAGGCET | 76 |
| BC-062 | CTGCCCCGGGTTCCTCATTCTCZFGGTGAGGGTCTGCTGTACGGCCAAGGCET | 77 |
| BC-063 | CTGCCCCGGGTTCCTCATTCTCZECGGGTTCGACTGCTGTACGGCCAAGGCET | 78 |
| BC-064 | CTGCCCCGGGTTCCTCATTCTCZETGCTACACCCTGCTGTACGGCCAAGGCET | 79 |
| BC-065 | CTGCCCCGGGTTCCTCATTCTCZEGGATCAAGCCTGCTGTACGGCCAAGGCET | 80 |
| BC-066 | CTGCCCCGGGTTCCTCATTCTCZEATGTAATGTCTGCTGTACGGCCAAGGCET | 81 |
| BC-067 | CTGCCCCGGGTTCCTCATTCTCZETCCTTAGGGCTGCTGTACGGCCAAGGCET | 82 |
| BC-068 | CTGCCCCGGGTTCCTCATTCTCZECATTGACGACTGCTGTACGGCCAAGGCET | 83 |
| BC-069 | CTGCCCCGGGTTCCTCATTCTCZEATATGCTTTCTGCTGTACGGCCAAGGCET | 84 |
| BC-070 | CTGCCCCGGGTTCCTCATTCTCZECCCTACAGACTGCTGTACGGCCAAGGCET | 85 |
| BC-071 | CTGCCCCGGGTTCCTCATTCTCZFCAGGGAACGCTGCTGTACGGCCAAGGCET | 86 |
| BC-072 | CTGCCCCGGGTTCCTCATTCTCZFAGTGAATACCTGCTGTACGGCCAAGGCET | 87 |
| BC-073 | CTGCCCCGGGTTCCTCATTCTCZECAATGACGTCTGCTGTACGGCCAAGGCET | 88 |
| BC-074 | CTGCCCCGGGTTCCTCATTCTCZFGGACGCTGACTGCTGTACGGCCAAGGCET | 89 |
| BC-075 | CTGCCCCGGGTTCCTCATTCTCZETATCTGGGCCTGCTGTACGGCCAAGGCET | 90 |
| BC-076 | CTGCCCCGGGTTCCTCATTCTCZFAGTTTTAGGCTGCTGTACGGCCAAGGCET | 91 |
| BC-077 | CTGCCCCGGGTTCCTCATTCTCZFTCTGGTCTTCTGCTGTACGGCCAAGGCET | 92 |
| BC-078 | CTGCCCCGGGTTCCTCATTCTCZEGCAATCATCCTGCTGTACGGCCAAGGCET | 93 |
| BC-079 | CTGCCCCGGGTTCCTCATTCTCZFGTAGAATTACTGCTGTACGGCCAAGGCET | 94 |
| BC-080 | CTGCCCCGGGTTCCTCATTCTCZETTTACGGTGCTGCTGTACGGCCAAGGCET | 95 |
| BC-081 | CTGCCCCGGGTTCCTCATTCTCZEAACGTCATTCTGCTGTACGGCCAAGGCET | 96 |
| BC-082 | CTGCCCCGGGTTCCTCATTCTCZETGAAGGGAGCTGCTGTACGGCCAAGGCET | 97 |
| BC-083 | CTGCCCCGGGTTCCTCATTCTCZEGATGGCGTACTGCTGTACGGCCAAGGCET | 98 |
| BC-084 | CTGCCCCGGGTTCCTCATTCTCZECGGATGAACCTGCTGTACGGCCAAGGCET | 99 |
| BC-085 | CTGCCCCGGGTTCCTCATTCTCZEGAAAGCGTTCTGCTGTACGGCCAAGGCET | 100 |
| BC-086 | CTGCCCCGGGTTCCTCATTCTCZFGTACCAGGACTGCTGTACGGCCAAGGCET | 101 |
| BC-087 | CTGCCCCGGGTTCCTCATTCTCZFTAGCAAAGCCTGCTGTACGGCCAAGGCET | 102 |
| BC-088 | CTGCCCCGGGTTCCTCATTCTCZETTGATCATGCTGCTGTACGGCCAAGGCET | 103 |
| BC-089 | CTGCCCCGGGTTCCTCATTCTCZFGGCTGTCTACTGCTGTACGGCCAAGGCET | 104 |
| BC-090 | CTGCCCCGGGTTCCTCATTCTCZETGACCTACTCTGCTGTACGGCCAAGGCET | 105 |
| BC-091 | CTGCCCCGGGTTCCTCATTCTCZECGTATTGGGCTGCTGTACGGCCAAGGCET | 106 |
| BC-092 | CTGCCCCGGGTTCCTCATTCTCZFAGGGATTACCTGCTGTACGGCCAAGGCET | 107 |
| BC-093 | CTGCCCCGGGTTCCTCATTCTCZETTACGATGCCTGCTGTACGGCCAAGGCET | 108 |
| BC-094 | CTGCCCCGGGTTCCTCATTCTCZFTGGGTGTTTCTGCTGTACGGCCAAGGCET | 109 |
| BC-095 | CTGCCCCGGGTTCCTCATTCTCZEAGTCCGGCACTGCTGTACGGCCAAGGCET | 110 |
| BC-096 | CTGCCCCGGGTTCCTCATTCTCZFATCGAAGAGCTGCTGTACGGCCAAGGCET | 111 |

LEGEND:
E = G-3′phosphorothioate
Z = T-3′phosphorothioate

TABLE IV

| | | SEQ ID |
|---|---|---|
| Adaptors: | Sequence 5′>3′ | NO: |
| P1-Adaptor | CCACTACGCCTCCGCTTTCCTCTCTATGGGCAGTCGGTGAT | 129 |
| (top strand) | | |
| P1-Adaptor | ATCACCGACTGCCCATAGAGAGGAAAGCGGAGGCGTAGTGG*T*T | 130 |
| (bottom strand) | | |
| A adapter (top | GTCGGAGACACGCAGGGATGAGATGG*T*T | 131 |
| strand) | | |
| A adapter | CCATCTCATCCCTGCGTGTCTCCGAC | 132 |
| (bottom strand) | | |
| Barcoded A | XXXXGTCGGAGACACGCAGGGATGAGATGG*T*T | 133 |
| adapter (top | | |
| strand) | | |
| Barcoded A | CCATCTCATCCCTGCGTGTCTCCGACTCAGXXXXXXXXXXAGT | 134 |
| adapter (bottom | | |
| strand) | | |
| Blocking oligo | ACTXXXXXXXXXXCTGAGTCGGAGACACGC | 135 |
| 1 | | |
| Blocking oligo | ATCXXXXXXXXXXCTGAGTCGGAGACACGCAGGGATGAGATGG | 136 |
| 2 | | |
| Blocking oligo | CTGAGTCGGAGACACGC | 137 |
| 3 | | |
| Blocking oligo | CTGAGTCGGAGACACGCAGGGATGAGATGG | 138 |
| 4 | | |
| A adapter (top | TTCCATCTCATCCCTGCGTGTCTCCGACTCAG | 140 |
| strand) | | |
| A adapter | CTGAGTCGGAGACACGCAGGGATGAGATGGAATT | 141 |
| (bottom strand) | | |

LEGEND:
*T = T-3′phosphorothioate
X = can be any of A, C, G or T

TABLE V

| SEQ ID | | |
|---|---|---|
| NO: | Blocker Oligo: | Sequences in 5′ to 3′ direction |
| 112 | P1 Blocker | ATCACCGACTGCCCATAGAGAGGAAAGCGGAGGCGTAGTGG |
| 113 | Barcode-001 | CGCCTTGGCCGTACAGCAGCCTCTTACACAGAGAATGAGGAACCCGGGGCAG |
| | Blocker | |
| 114 | Barcode-002 | CGCCTTGGCCGTACAGCAGACCACTCCCTAGAGAATGAGGAACCCGGGGCAG |
| | Blocker | |
| 115 | Barcode-003 | CGCCTTGGCCGTACAGCAGTATAACCTATAGAGAATGAGGAACCCGGGGCAG |
| | Blocker | |
| 116 | Barcode-004 | CGCCTTGGCCGTACAGCAGGACCGCATCCAGAGAATGAGGAACCCGGGGCAG |
| | Blocker | |
| 117 | Barcode-005 | CGCCTTGGCCGTACAGCAGCTTACACCACAGAGAATGAGGAACCCGGGGCAG |
| | Blocker | |
| 118 | Barcode-006 | CGCCTTGGCCGTACAGCAGTGTCCCTCGCAGAGAATGAGGAACCCGGGGCAG |
| | Blocker | |
| 119 | Barcode-007 | CGCCTTGGCCGTACAGCAGGGCATAACCCAGAGAATGAGGAACCCGGGGCAG |
| | Blocker | |
| 120 | Barcode-008 | CGCCTTGGCCGTACAGCAGATCCTCGCTCAGAGAATGAGGAACCCGGGGCAG |
| | Blocker | |
| 121 | Barcode-009 | CGCCTTGGCCGTACAGCAGGTCGCAACCTAGAGAATGAGGAACCCGGGGCAG |
| | Blocker | |
| 122 | Barcode-010 | CGCCTTGGCCGTACAGCAGAGCTTACCGCAGAGAATGAGGAACCCGGGGCAG |
| | Blocker | |
| 123 | Barcode-011 | CGCCTTGGCCGTACAGCAGCGTGTCGCACAGAGAATGAGGAACCCGGGGCAG |
| | Blocker | |
| 124 | Barcode-012 | CGCCTTGGCCGTACAGCAGTTTTCCTCTTAGAGAATGAGGAACCCGGGGCAG |
| | Blocker | |
| 125 | Barcode-013 | CGCCTTGGCCGTACAGCAGGCCTTACCGCAGAGAATGAGGAACCCGGGGCAG |
| | Blocker | |
| 126 | Barcode-014 | CGCCTTGGCCGTACAGCAGTCTGCCGCACAGAGAATGAGGAACCCGGGGCAG |
| | Blocker | |
| 127 | Barcode-015 | CGCCTTGGCCGTACAGCAGCATTCAACTCAGAGAATGAGGAACCCGGGGCAG |
| | Blocker | |
| 128 | Barcode-016 | CGCCTTGGCCGTACAGCAGAACGTCTCCCAGAGAATGAGGAACCCGGGGCAG |
| | Blocker | |
| 139 | A Blocker | CTGAGTCGGAGACACGCAGGGATGAGATGG |

TABLE VI

| Blocker Oligo- | | SEQ ID |
|---|---|---|
| nucleotides | Sequence 5′>3′ | NOS: |
| Blocker A | CTGAGTCGGAGACACGCAGGGATGAGATGG | 142 |
| Blocker P1 | ATCACCGACTGCCCATAGAGAGGAAAGCGGAGGCGTAGTGG | 143 |
| Blocker BC 1 | ATCGTTACCTTAGCTGAGTCGGAGACACGCAGGGATGAGATGG | 144 |
| Blocker BC 2 | ATCGTTCTCCTTACTGAGTCGGAGACACGCAGGGATGAGATGG | 145 |
| Blocker BC 3 | ATCGAATCCTCTTCTGAGTCGGAGACACGCAGGGATGAGATGG | 146 |
| Blocker BC 4 | ATCGATCTTGGTACTGAGTCGGAGACACGCAGGGATGAGATGG | 147 |
| Blocker BC 5 | ATCGTTCCTTCTGCTGAGTCGGAGACACGCAGGGATGAGATGG | 148 |
| Blocker BC 6 | ATCGAACTTGCAGCTGAGTCGGAGACACGCAGGGATGAGATGG | 149 |
| Blocker BC 7 | ATCGAATCACGAACTGAGTCGGAGACACGCAGGGATGAGATGG | 150 |
| Blocker BC 8 | ATCGTTATCGGAACTGAGTCGGAGACACGCAGGGATGAGATGG | 151 |
| Blocker BC 9 | ATCGTTCCGCTCACTGAGTCGGAGACACGCAGGGATGAGATGG | 152 |
| Blocker BC 10 | ATCGTTCGGTCAGCTGAGTCGGAGACACGCAGGGATGAGATGG | 153 |
| Blocker BC 11 | ATCGATTCGAGGACTGAGTCGGAGACACGCAGGGATGAGATGG | 154 |
| Blocker BC 12 | ATCGAACCACCTACTGAGTCGGAGACACGCAGGGATGAGATGG | 155 |
| Blocker BC 13 | ATCGTCCGTTAGACTGAGTCGGAGACACGCAGGGATGAGATGG | 156 |
| Blocker BC 14 | ATCGACACTCCAACTGAGTCGGAGACACGCAGGGATGAGATGG | 157 |
| Blocker BC 15 | ATCGACCTCTAGACTGAGTCGGAGACACGCAGGGATGAGATGG | 158 |
| Blocker BC 16 | ATCGTCATCCAGACTGAGTCGGAGACACGCAGGGATGAGATGG | 159 |
| Blocker BC 17 | ATCGACGAATAGACTGAGTCGGAGACACGCAGGGATGAGATGG | 160 |
| Blocker BC 18 | ATCGCAATTGCCTCTGAGTCGGAGACACGCAGGGATGAGATGG | 161 |
| Blocker BC 19 | ATCGTCCGACTAACTGAGTCGGAGACACGCAGGGATGAGATGG | 162 |
| Blocker BC 20 | ATCGATGGATCTGCTGAGTCGGAGACACGCAGGGATGAGATGG | 163 |
| Blocker BC 21 | ATCGTAATTGCGACTGAGTCGGAGACACGCAGGGATGAGATGG | 164 |
| Blocker BC 22 | ATCGCGTCTCGAACTGAGTCGGAGACACGCAGGGATGAGATGG | 165 |
| Blocker BC 23 | ATCGTTCGTGGCACTGAGTCGGAGACACGCAGGGATGAGATGG | 166 |
| Blocker BC 24 | ATCGAATGAGGTTCTGAGTCGGAGACACGCAGGGATGAGATGG | 167 |
| Blocker BC 25 | ATCGTATCTCAGGCTGAGTCGGAGACACGCAGGGATGAGATGG | 168 |
| Blocker BC 26 | ATCGAGGTTGTAACTGAGTCGGAGACACGCAGGGATGAGATGG | 169 |
| Blocker BC 27 | ATCGCGGATGGTTCTGAGTCGGAGACACGCAGGGATGAGATGG | 170 |
| Blocker BC 28 | ATCGATTCCGGATCTGAGTCGGAGACACGCAGGGATGAGATGG | 171 |
| Blocker BC 29 | ATCGAGTGGTCGACTGAGTCGGAGACACGCAGGGATGAGATGG | 172 |
| Blocker BC 30 | ATCGATAACCTCGCTGAGTCGGAGACACGCAGGGATGAGATGG | 173 |
| Blocker BC 31 | ATCGCAGCTTGGACTGAGTCGGAGACACGCAGGGATGAGATGG | 174 |
| Blocker BC 32 | ATCGTGTGTAAGACTGAGTCGGAGACACGCAGGGATGAGATGG | 175 |
| Blocker BC 33 | ATCGTTCAATGAGAACTGAGTCGGAGACACGCAGGGATGAGATGG | 176 |
| Blocker BC 34 | ATCGAACGATGCGACTGAGTCGGAGACACGCAGGGATGAGATGG | 177 |
| Blocker BC 35 | ATCGACAATGGCTTACTGAGTCGGAGACACGCAGGGATGAGATGG | 178 |
| Blocker BC 36 | ATCGACGATTCCTTCTGAGTCGGAGACACGCAGGGATGAGATGG | 179 |
| Blocker BC 37 | ATCGACATTCTCAAGCTGAGTCGGAGACACGCAGGGATGAGATGG | 180 |
| Blocker BC 38 | ATCGTCCGTCCTCCACTGAGTCGGAGACACGCAGGGATGAGATGG | 181 |
| Blocker BC 39 | ATCGCCGATTGTTACTGAGTCGGAGACACGCAGGGATGAGATGG | 182 |
| Blocker BC 40 | ATCGATTATGTCAGCTGAGTCGGAGACACGCAGGGATGAGATGG | 183 |
| Blocker BC 41 | ATCGCGAAGTGGAACTGAGTCGGAGACACGCAGGGATGAGATGG | 184 |
| Blocker BC 42 | ATCGATTCGTGCTCTGAGTCGGAGACACGCAGGGATGAGATGG | 185 |
| Blocker BC 43 | ATCGCGGTGTCAAGCTGAGTCGGAGACACGCAGGGATGAGATGG | 186 |
| Blocker BC 44 | ATCGCTGGCCTCCAACTGAGTCGGAGACACGCAGGGATGAGATGG | 187 |
| Blocker BC 45 | ATCGAGGAAGCTCCACTGAGTCGGAGACACGCAGGGATGAGATGG | 188 |
| Blocker BC 46 | ATCGTTCGGACTGACTGAGTCGGAGACACGCAGGGATGAGATGG | 189 |
| Blocker BC 47 | ATCGTGGTTGCCTTACTGAGTCGGAGACACGCAGGGATGAGATGG | 190 |
| Blocker BC 48 | ATCGTCTCTTAGAACTGAGTCGGAGACACGCAGGGATGAGATGG | 191 |
| Blocker BC 49 | ATCGTTATGTTAGGACTGAGTCGGAGACACGCAGGGATGAGATGG | 192 |
| Blocker BC 50 | ATCGCCATTGTCCGCTGAGTCGGAGACACGCAGGGATGAGATGG | 193 |
| Blocker BC 51 | ATCGAATAGGCTCAACTGAGTCGGAGACACGCAGGGATGAGATGG | 194 |
| Blocker BC 52 | ATCGTTCCATGCGGCTGAGTCGGAGACACGCAGGGATGAGATGG | 195 |
| Blocker BC 53 | ATCGAGGATTGCCAGCTGAGTCGGAGACACGCAGGGATGAGATGG | 196 |
| Blocker BC 54 | ATCGCGATTCTCCGGCTGAGTCGGAGACACGCAGGGATGAGATGG | 197 |
| Blocker BC 55 | ATCGAGGAGGTGGACTGAGTCGGAGACACGCAGGGATGAGATGG | 198 |
| Blocker BC 56 | ATCGAATTAATGCTGCTGAGTCGGAGACACGCAGGGATGAGATGG | 199 |
| Blocker BC 57 | ATCGCCGTTGCCAGACTGAGTCGGAGACACGCAGGGATGAGATGG | 200 |
| Blocker BC 58 | ATCGTGTTCTAGGACTGAGTCGGAGACACGCAGGGATGAGATGG | 201 |
| Blocker BC 59 | ATCGAACATCAAGGACTGAGTCGGAGACACGCAGGGATGAGATGG | 202 |
| Blocker BC 60 | ATCGAAGAGCTAGACTGAGTCGGAGACACGCAGGGATGAGATGG | 203 |
| Blocker BC 61 | ATCGATCCGAGTGACTGAGTCGGAGACACGCAGGGATGAGATGG | 204 |
| Blocker BC 62 | ATCGTGAAGCAGGAACTGAGTCGGAGACACGCAGGGATGAGATGG | 205 |
| Blocker BC 63 | ATCGAACTCTAAGGCTGAGTCGGAGACACGCAGGGATGAGATGG | 206 |
| Blocker BC 64 | ATCGTCGGAACTCAGCTGAGTCGGAGACACGCAGGGATGAGATGG | 207 |
| Blocker BC 65 | ATCGATGTGCCAGGACTGAGTCGGAGACACGCAGGGATGAGATGG | 208 |
| Blocker BC 66 | ATCGATGATTGCGGCTGAGTCGGAGACACGCAGGGATGAGATGG | 209 |
| Blocker BC 67 | ATCGACTGGTAGGAACTGAGTCGGAGACACGCAGGGATGAGATGG | 210 |
| Blocker BC 68 | ATCGAACTTCTTGACTGAGTCGGAGACACGCAGGGATGAGATGG | 211 |
| Blocker BC 69 | ATCGCCAATTGAACTGAGTCGGAGACACGCAGGGATGAGATGG | 212 |
| Blocker BC 70 | ATCGACCAGTAGGCTGAGTCGGAGACACGCAGGGATGAGATGG | 213 |
| Blocker BC 71 | ATCGTCGGAGCCTCACTGAGTCGGAGACACGCAGGGATGAGATGG | 214 |
| Blocker BC 72 | ATCGTGTGGCCTTCGCTGAGTCGGAGACACGCAGGGATGAGATGG | 215 |
| Blocker BC 73 | ATCGACAGGCAGACTGAGTCGGAGACACGCAGGGATGAGATGG | 216 |
| Blocker BC 74 | ATCGAACCGATCGCTGAGTCGGAGACACGCAGGGATGAGATGG | 217 |
| Blocker BC 75 | ATCGTATTCCTGACTGAGTCGGAGACACGCAGGGATGAGATGG | 218 |
| Blocker BC 76 | ATCGAGGTTCTTCCGCTGAGTCGGAGACACGCAGGGATGAGATGG | 219 |
| Blocker BC 77 | ATCGAATCGCTTCGCTGAGTCGGAGACACGCAGGGATGAGATGG | 220 |
| Blocker BC 78 | ATCGAGAATTGGCTGCTGAGTCGGAGACACGCAGGGATGAGATGG | 221 |
| Blocker BC 79 | ATCGACAACCAGGCTGAGTCGGAGACACGCAGGGATGAGATGG | 222 |
| Blocker BC 80 | ATCGCCTGCCTTCGACTGAGTCGGAGACACGCAGGGATGAGATGG | 223 |
| Blocker BC 81 | ATCGCGAATGGCAGGCTGAGTCGGAGACACGCAGGGATGAGATGG | 224 |
| Blocker BC 82 | ATCGAGATGCCAACTGAGTCGGAGACACGCAGGGATGAGATGG | 225 |
| Blocker BC 83 | ATCGAATGTCCTAGCTGAGTCGGAGACACGCAGGGATGAGATGG | 226 |
| Blocker BC 84 | ATCGTTATGGAAGCTGAGTCGGAGACACGCAGGGATGAGATGG | 227 |
| Blocker BC 85 | ATCGTTGAGGCTGGCTGAGTCGGAGACACGCAGGGATGAGATGG | 228 |
| Blocker BC 86 | ATCGAATAACCAAGCTGAGTCGGAGACACGCAGGGATGAGATGG | 229 |
| Blocker BC 87 | ATCGTCCAGCCAACTGAGTCGGAGACACGCAGGGATGAGATGG | 230 |
| Blocker BC 88 | ATCGAAGTGTTCGGCTGAGTCGGAGACACGCAGGGATGAGATGG | 231 |
| Blocker BC 89 | ATCGAGATTCAGGACTGAGTCGGAGACACGCAGGGATGAGATGG | 232 |
| Blocker BC 90 | ATCGCCGTGGTTAGCTGAGTCGGAGACACGCAGGGATGAGATGG | 233 |
| Blocker BC 91 | ATCGCATCCTTCCGCTGAGTCGGAGACACGCAGGGATGAGATGG | 234 |
| Blocker BC 92 | ATCGCGGTTCCTAGCTGAGTCGGAGACACGCAGGGATGAGATGG | 235 |
| Blocker BC 93 | ATCGATTGGACAAGCTGAGTCGGAGACACGCAGGGATGAGATGG | 236 |
| Blocker BC 94 | ATCGCTTGTCGGACTGAGTCGGAGACACGCAGGGATGAGATGG | 237 |
| Blocker BC 95 | ATCGATCTGTCCGCTGAGTCGGAGACACGCAGGGATGAGATGG | 238 |
| Blocker BC 96 | ATCGACCGCTTAACTGAGTCGGAGACACGCAGGGATGAGATGG | 239 |

### Capture Oligonucleotides

In some embodiments, the present teachings provide compositions and methods for capturing target polynucleotides, where capture oligonucleotides can comprise an oligonucleotide. In some embodiments, capture oligonucleotides can be DNA, cDNA, RNA, or RNA/DNA hybrids. Capture oligonucleotides can be single-stranded or double-stranded nucleic acids or analogs thereof.

In some embodiments, capture oligonucleotides can include nucleotide sequences that can hybridize to any portion of a target polynucleotide. In some embodiments, capture oligonucleotides can include nucleotide sequences that are fully or partially complementary to any portion of a target polynucleotide. For example, capture oligonucleotides can include sequences that are complementary to chromosomal, genomic, organellar (e.g., mitochondrial, chloroplast or ribosomal), recombinant molecules, cloned, amplified (e.g., PCR amplified), cDNA, RNA such as precursor mRNA or mRNA, oligonucleotide, or any type of nucleic acid library. In some embodiments, capture oligonucleotides can include sequences that are complementary to any sequence from any organism such as prokaryotes, eukaryotes (e.g., humans, plants and animals), fungus, or viruses. In some embodiments, capture oligonucleotides can include sequences that are complementary to any sequence from normal or diseased cells or tissues or organs. In some embodiments, when multiple capture oligonucleotides hybridize to a target polynucleotide, they can hybridize to regions that overlap or are not overlapping.

In some embodiments, capture oligonucleotides can include nucleotide sequences that are fully complementary (e.g., base pairing A-T and/or C/G) or partially complementary (e.g., mis-match pairing A/C or G, T/C or G, C/A or T, or G/A or T) to any portion of the polynucleotide constructs. In some embodiments, capture oligonucleotides can include degenerate sequences. In some embodiments, capture oligonucleotides can include one or more inosine residues.

Capture oligonucleotides can be any length, including about 5-25 bases, or about 25-50 bases, or about 50-75 bases, or about 75-100 bases, or about 100-125 bases, or about 125-150 bases, or about 150-175 bases, or about 175-200 bases, or about 200-225 bases, or about 225-250 bases, or about 250-275 bases, or about 275-300 bases, or about 300-500 bases, or longer.

In some embodiments, polynucleotides can be hybridized with about 500,000-1 million different capture oligonucleotides, or with about 1-1.5 million different capture oligonucleotides, or with about 1.5-2 million different capture oligonucleotides, or with about 2-2.5 million different capture oligonucleotides, or with about 2.5-3 million different capture oligonucleotides, or more.

In some embodiments, capture oligonucleotides can include at least one scissile linkage. In some embodiments, a scissile linkage can be susceptible to cleavage or degradation by an enzyme or chemical compound. In some embodiments, capture oligonucleotides can include at least one phosphorothiolate, phosphorothioate, and/or phosphoramidate linkage.

### Binding Partners

In some embodiments, the present teachings provide compositions and methods for capturing target polynucleotides, where capture oligonucleotides can include one member of a binding partner. In some embodiments, molecules that function as binding partners include: biotin (and its derivatives) and their binding partner avidin moieties, streptavidin moieties (and their derivatives); His-tags which bind with nickel, cobalt or copper; cysteine, histidine, or histidine patch which bind Ni-NTA; maltose which binds with maltose binding protein (MBP); lectin-carbohydrate binding partners; calcium-calcium binding protein (CBP); acetylcholine and receptor-acetylcholine; protein A and binding partner anti-FLAG antibody; GST and binding partner glutathione; uracil DNA glycosylase (UDG) and ugi (uracil-DNA glycosylase inhibitor) protein; antigen or epitope tags which bind to antibody or antibody fragments, particularly antigens such as digoxigenin, fluorescein, dinitrophenol or bromodeoxyuridine and their respective antibodies; mouse immunoglobulin and goat anti-mouse immunoglobulin; IgG bound and protein A; receptor-receptor agonist or receptor antagonist; enzyme-enzyme cofactors; enzyme-enzyme inhibitors; and thyroxine-cortisol. Another binding partner for biotin can be a biotin-binding protein from chicken (Hytonen, et al., BMC Structural Biology 7:8).

An avidin moiety can include an avidin protein, as well as any derivatives, analogs and other non-native forms of avidin that can bind to biotin moieties. Other forms of avidin moieties include native and recombinant avidin and streptavidin as well as derivatized molecules, e.g. nonglycosylated avidins, N-acyl avidins and truncated streptavidins. For example, avidin moiety includes deglycosylated forms of avidin, bacterial streptavidins produced by Streptomyces (e.g., Streptomyces avidinii), truncated streptavidins, recombinant avidin and streptavidin as well as to derivatives of native, deglycosylated and recombinant avidin and of native, recombinant and truncated streptavidin, for example, N-acyl avidins, e.g., N-acetyl, N-phthalyl and N-succinyl avidin, and the commercial products ExtrAvidin™, Captavidin™, Neutravidin™ and Neutralite Avidin™.

### Surfaces

In some embodiments, the present teachings provide compositions and methods for capturing target polynucleotides, where capture oligonucleotides that are attached to a member of a binding partner (e.g., biotin) can bind another member of a binding partner (e.g., avidin-like, such as streptavidin) which is attached to a surface. In some embodiments, a surface can be an outer or top-most layer or boundary of an object. In some embodiments, a surface can be interior to the boundary of an object. In some embodiments, a surface can be porous or non-porous. In some embodiments, a surface can be a planar surface, as well as concave, convex, or any combination thereof. In some embodiments, a surface can be a bead, particle, microparticle, sphere, filter, flowcell, or gel. In some embodiments, a surface includes the inner walls of a capillary, a channel, a well, groove, channel, reservoir. In some embodiments, a surface can include texture (e.g., etched, cavitated, pores, three-dimensional scaffolds or bumps). In some embodiments, a surface can be made from materials such as glass, borosilicate glass, silica, quartz, fused quartz, mica, polyacrylamide, plastic polystyrene, polycarbonate, polymethacrylate (PMA), polymethyl methacrylate (PMMA), polydimethylsiloxane (PDMS), silicon, germanium, graphite, ceramics, silicon, semiconductor, high refractive index dielectrics, crystals, gels, polymers, or films (e.g., films of gold, silver, aluminum, or diamond). In some embodiments, a surface can be magnetic or paramagnetic (e.g., magnetic or paramagnetic microparticles). In some embodiments, paramagnetic microparticles can be paramagnetic beads attached with streptavidin (e.g., Dynabeads™ M-270 from Invitrogen, Carlsbad, Calif.).

### Additional Reactions

In some embodiments, the present teachings provide compositions and methods for capturing target polynucleotides, where capture duplexes, enriched target polynucleotides and/or released target polynucleotides can be subjected to further manipulations. In some embodiments, further manipulations can include nucleic acid manipulations. Nucleic acid manipulation can be conducted in any combination and in any order and include: chemical modification, size-selection, end repairing, tailing, adaptor-joining, ligation, nick repairing, purification, nick translation, amplification, surface attachment and/or sequencing. In some embodiments, any of these nucleic acid manipulations can be omitted or can be repeated.

### Chemical Modifications

In some embodiments, reduced complexity target polynucleotides can be modified to attach to a surface. For example, reduced complexity target polynucleotides can be amino-modified for attachment to a surface (e.g., particles or a planar surface). In some embodiments, an amino-modified nucleic acid can be attached to a surface that is coated with a carboxylic acid. In some embodiments, an amino-modified nucleic acid can be reacted with EDC (or EDAC) for attachment to a carboxylic acid coated surface (with or without NHS). In some embodiments, target polynucleotides can be attached to particles, such as Ion Sphere™ particles (Life Technologies).

### Amplification

In some embodiments, reduced complexity target polynucleotides can be amplified. In some embodiments, amplification can be conducted using at least one amplification primer that can hybridize to either strand or any portion of the polynucleotide constructs, including a nucleic acid adaptor or a target polynucleotide. In some embodiments, amplification can include thermo-cycling amplification or isothermal amplification reactions. In some embodiments, amplification can be conducted with polymerase that are thermo-stable or thermo-labile. In some embodiments, amplification can be conducted as a PCR reaction.

### Size-Selection:

In some embodiments, reduced complexity target polynucleotides can be subjected to any size-selection procedure to obtain any desired size range. In some embodiments, reduced complexity target polynucleotides are not size-selected.

In some embodiments, nucleic acid size selection method includes without limitation: solid phase adherence or immobilization; electrophoresis, such as gel electrophoresis; and chromatography, such as HPLC and size exclusion chromatography. In some embodiments, a solid phase adherence/immobilization methods involves paramagnetic beads coated with a chemical functional group that interacts with nucleic acids under certain ionic strength conditions with or without polyethylene glycol or polyalkylene glycol.

Examples of solid phase adherence/immobilization methods include but are not limited to: SPRI (Solid Phase Reversible Immobilization) beads from Agencourt (see Hawkins 1995 Nucleic Acids Research 23:22) which are carboxylate-modified paramagnetic beads; MAGNA PURE magnetic glass particles (Roche Diagnostics, Hoffmann-La Roche Ltd.); MAGNESIL magnetic bead kit from Promega; BILATEST magnetic bead kit from Bilatec AG; MAGTRATION paramagnetic system from Precision System Science, Inc.; MAG BIND from Omega Bio-Tek; MAGPREP silica from Merck/Estapor; SNARe DNA purification system from Bangs; CHEMAGEN M-PVA beads from CHEMAGEN; and magnetic beads from Aline Bioscience (DNA Purification Kit).

In some embodiments, size-selected nucleic acids can be about 50-250 bp, or about 250-500 bp, or about 500-750 bp, or about 750-1000 bp, or about 1-5 kb, or about 5-10 kb, or about 10-25 kb, or about 25-50 kb, or about 50-60 kb or longer.

### Repairing Nucleic Acid Fragments:

In some embodiments, repairing reduced complexity target polynucleotides may be desirable. In some embodiments, reduced complexity target polynucleotides can have a first end, a second end, or an internal portion, having undesirable features, such as nicks, overhang ends, ends lacking a phosphorylated end, ends having a phosphorylated end, or nucleic acid fragments having apurinic or apyrimidinic residues. In some embodiments, enzymatic reactions can be conducted to repair one or more ends or internal portions. In some embodiments, reduced complexity target polynucleotides can be subjected to enzymatic reactions to convert overhang ends to blunt ends, or to phosphorylate or de-phosphorylate the 5′ end of a strand, or to close nicks, to repair oxidized purines or pyrimidines, to repair deaminated cytosines, or to hydrolyze the apurinic or apyrimidinic residues. In some embodiments, repairing or end-repairing target polynucleotides includes contacting nucleic acid fragments with: an enzyme to close single-stranded nicks in duplex DNA (e.g., T4 DNA ligase); an enzyme to phosphorylate the 5′ end of at least one strand of a duplex DNA (e.g., T4 polynucleotide kinase); an enzyme to remove a 5′ or 3′phosphate (e.g., any phosphatase enzyme, such as calf intestinal alkaline phosphatase, bacterial alkaline phosphatase, shrimp alkaline phosphatase, Antarctic phosphatase, and placental alkaline phosphatase); an enzyme to remove 3′ overhang ends (e.g., DNA polymerase I, Large (Klenow) fragment, T4 DNA polymerase, mung bean nuclease); an enzyme to fill-in 5′ overhang ends (e.g., T4 DNA polymerase, Tfi DNA polymerase, Tli DNA polymerase, Taq DNA polymerase, Large (Klenow) fragment, phi29 DNA polymerase, Mako DNA polymerase (Enyzmatics, Beverly, Mass.), or any heat-stable or heat-labile DNA polymerase); an enzyme to remove 5′ overhang ends (e.g., S1 nuclease); an enzyme to remove 5′ or 3′ overhang ends (e.g., mung bean nuclease); an enzyme to hydrolyze single-stranded DNA (e.g., nuclease P1); an enzyme to remove both strands of double-stranded DNA (e.g., nuclease Bal-31); and/or an enzyme to remove an apurinic or apyrimidinic residue (e.g., endonuclease IV). In some embodiments, the polymerases can have exonuclease activity, or have a reduced or lack exonuclease activity.

In some embodiments, a repairing or end-repairing reaction can be supplemented with additional repairing enzymes in any combination and in any amount, including: endonuclease IV (apurinic-apyrimidinic removal), Bst DNA polymerase (5′>3′ exonuclease for nick translation), formamidopyrimidine DNA glycosylase (FPG) (e.g., base excision repair for oxidize purines), uracil DNA glycosylase (uracil removal), T4 endonuclease V (pyrimidine removal) and/or endonuclease VIII (removes oxidized pyrimidines). In some embodiments, a repairing or end-repairing reaction can be conducted in the presence of appropriate co-factors, including dNTPs, NAD, (NH₄)₂SO₄, KCl, and/or MgSO₄.

### Purification Steps:

In some embodiments, reduced complexity target polynucleotides can be subjected to any purification procedure to remove non-desirable materials (buffers, salts, enzymes, primer-dimers, or excess adaptors or primers). In some embodiments, a purification procedure can be conducted between any two steps to remove buffers, salts, enzymes, adaptors, non-reacted nucleic acid fragments, and the like. Purification procedures include without limitation: bead purification, column purification, gel electrophoresis, dialysis, alcohol precipitation, and size-selective PEG precipitation.

### Tailing

In some embodiments, reduced complexity target polynucleotides can be subjected to a tailing reaction (e.g., non-template-dependent terminal transferase reaction). In some embodiments, a non-template-dependent terminal transferase reaction can be catalyzed by a Taq polymerase, Tfi DNA polymerase, 3′ exonuclease minus-large (Klenow) fragment, or 3′ exonuclease minus-T4 polymerase.

### Nick Repair

In some embodiments, reduced complexity target polynucleotides can be subject to a nick repairing or nick repair reaction. In some embodiments, a nick repair reaction can be catalyzed by a nick repair polymerase such as Taq DNA polymerase, Bst DNA polymerase, Platinum® Pfx DNA polymerase (Invitrogen), Tfi Exo(−) DNA polymerase (Invitrogen) or Phusion® Hot Start High-Fidelity DNA polymerase (New England Biolabs). In some embodiments, the nick repair enzyme can be used to extend the nucleic acid strand from the site of the nick to the original termini of the adaptor sequence.

### Labeled Nucleotides

In some embodiments, nucleotides (or analogs thereof) used for any nucleic acid manipulation can be attached to a label. In some embodiments, a label comprises a detectable moiety. In some embodiments, a label can generate, or cause to generate, a detectable signal. A detectable signal can be generated from a chemical or physical change (e.g., heat, light, electrical, pH, salt concentration, enzymatic activity, or proximity events). For example, a proximity event can include two reporter moieties approaching each other, or associating with each other, or binding each other. A detectable signal can be detected optically, electrically, chemically, enzymatically, thermally, or via mass spectroscopy or Raman spectroscopy. A label can include compounds that are luminescent, photoluminescent, electroluminescent, bioluminescent, chemiluminescent, fluorescent, phosphorescent or electrochemical. A label can include compounds that are fluorophores, chromophores, radioisotopes, haptens, affinity tags, atoms or enzymes. In some embodiments, the label comprises a moiety not typically present in naturally occurring nucleotides. For example, the label can include fluorescent, luminescent or radioactive moieties.

### Sequencing Reactions

In some embodiments, reduced complexity target polynucleotides can be sequenced by any sequencing method, including sequencing-by-synthesis, ion-based sequencing involving the detection of sequencing byproducts using field effect transistors (e.g., FETs and ISFETs), chemical degradation sequencing, ligation-based sequencing, hybridization sequencing, pyrophosphate detection sequencing, capillary electrophoresis, gel electrophoresis, next-generation, massively parallel sequencing platforms, sequencing platforms that detect hydrogen ions or other sequencing by-products, and single molecule sequencing platforms. In some embodiments, a sequencing reaction can be conducted using at least one sequencing primer that can hybridize to any portion of the polynucleotide constructs, including a nucleic acid adaptor or a target polynucleotide.

### Workflows

In some embodiments, reduced complexity target polynucleotides produced by the methods described herein can be used in any nucleic acid sequencing workflow, including sequencing by oligonucleotide probe ligation and detection (e.g., SOLiD™ from Life Technologies, WO 2006/084131), probe-anchor ligation sequencing (e.g., Complete Genomics™ or Polonator™), sequencing-by-synthesis (e.g., Genetic Analyzer and HiSeq™, from Illumina), pyrophosphate sequencing (e.g., Genome Sequencer FLX from 454 Life Sciences), ion-sensitive sequencing (e.g., Personal Genome Machine (PGM™) and Ion Proton™ Sequencer, both from Ion Torrent Systems, Inc.), and single molecule sequencing platforms (e.g., HeliScope™ from Helicos™).

In some embodiments, genomic DNA can be isolated from a cell, tissue or organ. In some embodiments, genomic DNA can be fragmented via enzymatic, chemical or physical fragmentation methods. In some embodiments, fragmented DNA (e.g., polynucleotides) can be joined to at least one nucleic acid adaptor to form a polynucleotide library. In some embodiments, a collection of non-target and target polynucleotide constructs can form a nucleic acid library. In some embodiments, a nucleic acid library can be amplified. In some embodiments, a nucleic acid library can be denatured to form a single-stranded library. In some embodiments, a single stranded nucleic acid library can be hybridized to at least one blocker oligonucleotide and at least one biotinylated capture oligonucleotide and non-specific oligonucleotides (e.g., human Cot-1 DNA), under suitable hybridization conditions to form capture duplexes having target polynucleotides hybridized to capture oligonucleotides. For example, suitable hybridization conditions can include about 40-50° C. for about 60-75 hours. In some embodiments, paramagnetic streptavidin beads can be reacted with the capture duplexes to recover the enriched target polynucleotides. For example, the paramagnetic streptavidin beads and capture duplexes can be reacted at about 40-50° C. for about 15-75 minutes to form a bead-duplex complex. In some embodiments, the bead-duplex complex can be washed with a buffer (e.g., high stringency wash buffer) to remove un-hybridized nucleic acids to enrich for target polynucleotides hybridized to biotinylated capture oligonucleotides. In some embodiments, the enriched target polynucleotides can be denatured to release single-stranded target polynucleotides from the beaded capture oligonucleotides, or can remain bound to the beaded capture oligonucleotides. In some embodiments, enriched target polynucleotides can be amplified. In some embodiments, amplified target polynucleotides can be conjugated to microparticles and amplified to form microparticles templated with clonal copies of the target polynucleotide. In some embodiments, target polynucleotides attached to the microparticles can be sequenced in any sequencing platform (e.g., Ion Torrent PGM™ or Proton™ sequencer (Ion Torrent™ Systems, Life Technologies Corporation).

### Ion Sensitive Sequencing Methods

In some embodiments, one or more reduced complexity target polynucleotides produced according to the present teachings can be sequenced using methods that detect one or more byproducts of nucleotide incorporation. The detection of polymerase extension by detecting physicochemical byproducts of the extension reaction, can include pyrophosphate, hydrogen ion, charge transfer, heat, and the like, as disclosed, for example, in Pourmand et al, Proc. Natl. Acad. Sci., 103: 6466-6470 (2006); Purushothaman et al., IEEE ISCAS, IV-169-172; Rothberg et al, U.S. Patent Publication No. 2009/0026082; Anderson et al, Sensors and Actuators B Chem., 129: 79-86 (2008); Sakata et al., Angew. Chem. 118:2283-2286 (2006); Esfandyapour et al., U.S. Patent Publication No. 2008/01666727; and Sakurai et al., Anal. Chem. 64: 1996-1997 (1992).

Reactions involving the generation and detection of ions are widely performed. The use of direct ion detection methods to monitor the progress of such reactions can simplify many current biological assays. For example, template-dependent nucleic acid synthesis by a polymerase can be monitored by detecting hydrogen ions that are generated as natural byproducts of nucleotide incorporations catalyzed by the polymerase. Ion-sensitive sequencing (also referred to as “pH-based” or “ion-based” nucleic acid sequencing) exploits the direct detection of ionic byproducts, such as hydrogen ions, that are produced as a byproduct of nucleotide incorporation. In one exemplary system for ion-based sequencing, the nucleic acid to be sequenced can be captured in a microwell, and nucleotides can be flowed across the well, one at a time, under nucleotide incorporation conditions. The polymerase incorporates the appropriate nucleotide into the growing strand, and the hydrogen ion that is released can change the pH in the solution, which can be detected by an ion sensor that is coupled with the well. This technique does not require labeling of the nucleotides or expensive optical components, and allows for far more rapid completion of sequencing runs. Examples of such ion-based nucleic acid sequencing methods and platforms include the Ion Torrent PGM™ or Proton™ sequencer (Ion Torrent™ Systems, Life Technologies Corporation).

In some embodiments, target polynucleotides produced using the methods, systems and kits of the present teachings can be used as a substrate for a biological or chemical reaction that is detected and/or monitored by a sensor including a field-effect transistor (FET). In various embodiments the FET is a chemFET or an ISFET. A “chemFET” or chemical field-effect transistor, is a type of field effect transistor that acts as a chemical sensor. It is the structural analog of a MOSFET transistor, where the charge on the gate electrode is applied by a chemical process. An “ISFET” or ion-sensitive field-effect transistor, is used for measuring ion concentrations in solution; when the ion concentration (such as H+) changes, the current through the transistor will change accordingly. A detailed theory of operation of an ISFET is given in “Thirty years of ISFETOLOGY: what happened in the past 30 years and what may happen in the next 30 years,” P. Bergveld, Sens. Actuators, 88 (2003), pp. 1-20.

In some embodiments, the FET may be a FET array. As used herein, an “array” is a planar arrangement of elements such as sensors or wells. The array may be one or two dimensional. A one dimensional array can be an array having one column (or row) of elements in the first dimension and a plurality of columns (or rows) in the second dimension. The number of columns (or rows) in the first and second dimensions may or may not be the same. The FET or array can comprise 102, 103, 104, 105, 106, 107 or more FETs.

In some embodiments, one or more microfluidic structures can be fabricated above the FET sensor array to provide for containment and/or confinement of a biological or chemical reaction. For example, in one implementation, the microfluidic structure(s) can be configured as one or more wells (or microwells, or reaction chambers, or reaction wells, as the terms are used interchangeably herein) disposed above one or more sensors of the array, such that the one or more sensors over which a given well is disposed detect and measure analyte presence, level, and/or concentration in the given well. In some embodiments, there can be a 1:1 correspondence of FET sensors and reaction wells.

Microwells or reaction chambers are typically hollows or wells having well-defined shapes and volumes which can be manufactured into a substrate and can be fabricated using conventional microfabrication techniques, e.g. as disclosed in the following references: Doering and Nishi, Editors, Handbook of Semiconductor Manufacturing Technology, Second Edition (CRC Press, 2007); Saliterman, Fundamentals of BioMEMS and Medical Microdevices (SPIE Publications, 2006); Elwenspoek et al, Silicon Micromachining (Cambridge University Press, 2004); and the like. Examples of configurations (e.g. spacing, shape and volumes) of microwells or reaction chambers are disclosed in Rothberg et al, U.S. patent publication 2009/0127589; Rothberg et al, U.K. patent application GB24611127.

In some embodiments, the biological or chemical reaction can be performed in a solution or a reaction chamber that is in contact with or capacitively coupled to a FET such as a chemFET or an ISFET. The FET (or chemFET or ISFET) and/or reaction chamber can be an array of FETs or reaction chambers, respectively.

In some embodiments, a biological or chemical reaction can be carried out in a two-dimensional array of reaction chambers, wherein each reaction chamber can be coupled to a FET, and each reaction chamber is no greater than 10 μm³ (i.e., 1 pL) in volume. In some embodiments each reaction chamber is no greater than 0.34 pL, 0.096 pL or even 0.012 pL in volume. A reaction chamber can optionally be 22, 32, 42, 52, 62, 72, 82, 92, or 102 square microns in cross-sectional area at the top. Preferably, the array has at least 102, 103, 104, 105, 106, 107, 108, 109, or more reaction chambers. In some embodiments, the reaction chambers can be capacitively coupled to the FETs.

FET arrays as used in various embodiments according to the disclosure can be fabricated according to conventional CMOS fabrications techniques, as well as modified CMOS fabrication techniques and other semiconductor fabrication techniques beyond those conventionally employed in CMOS fabrication. Additionally, various lithography techniques can be employed as part of an array fabrication process.

Exemplary FET arrays suitable for use in the disclosed methods, as well as microwells and attendant fluidics, and methods for manufacturing them, are disclosed, for example, in U.S. Patent Publication No. 20100301398; U.S. Patent Publication No. 20100300895; U.S. Patent Publication No. 20100300559; U.S. Patent Publication No. 20100197507, U.S. Patent Publication No. 20100137143; U.S. Patent Publication No. 20090127589; and U.S. Patent Publication No. 20090026082, which are incorporated by reference in their entireties.

In one aspect, the disclosed methods, compositions, systems, apparatuses and kits can be used for carrying out label-free nucleic acid sequencing, and in particular, ion-based nucleic acid sequencing. The concept of label-free detection of nucleotide incorporation has been described in the literature, including the following references that are incorporated by reference: Rothberg et al, U.S. patent publication 2009/0026082; Anderson et al, Sensors and Actuators B Chem., 129: 79-86 (2008); and Pourmand et al, Proc. Natl. Acad. Sci., 103: 6466-6470 (2006). Briefly, in nucleic acid sequencing applications, nucleotide incorporations are determined by measuring natural byproducts of polymerase-catalyzed extension reactions, including hydrogen ions, polyphosphates, PPi, and Pi (e.g., in the presence of pyrophosphatase). Examples of such ion-based nucleic acid sequencing methods and platforms include the Ion Torrent PGM™ or Proton™ sequencer (Ion Torrent™ Systems, Life Technologies Corporation).

In some embodiments, the disclosure relates generally to methods for sequencing the reduced complexity target polynucleotides produced by the teachings provided herein. In one exemplary embodiment, the disclosure relates generally to a method for obtaining sequence information from reduced complexity target polynucleotides, comprising: (a) conducting reactions to obtain reduced complexity target polynucleotides; and (b) performing template-dependent nucleic acid synthesis using at least one of the reduced complexity target polynucleotides produced during step (a) as a template.

In some embodiments, capturing for target polynucleotides can include hybridizing a plurality of polynucleotide to one or more blocker oligonucleotides and with one or more capture oligonucleotides to form duplexes having a target polynucleotide hybridized to a capture oligonucleotide. In some embodiments, the methods can further comprise separating the duplexes from nucleic acids in the sample that are not part of a duplex to obtain enriched target polynucleotides.

In some embodiments, the template-dependent synthesis includes incorporating one or more nucleotides in a template-dependent fashion into a newly synthesized nucleic acid strand.

Optionally, the methods can further include producing one or more ionic byproducts of such nucleotide incorporation.

In some embodiments, the methods can further include detecting the incorporation of the one or more nucleotides into the sequencing primer. Optionally, the detecting can include detecting the release of hydrogen ions.

In another embodiment, the disclosure relates generally to a method for sequencing a nucleic acid, comprising: (a) producing a plurality of reduced complexity target polynucleotides according to the methods disclosed herein; (b) disposing a plurality of reduced complexity target polynucleotides into a plurality of reaction chambers, wherein one or more of the reaction chambers are in contact with a field effect transistor (FET). Optionally, the method further includes contacting at least one of the reduced complexity target polynucleotides disposed into one of the reaction chambers with a polymerase, thereby synthesizing a new nucleic acid strand by sequentially incorporating one or more nucleotides into a nucleic acid molecule. Optionally, the method further includes generating one or more hydrogen ions as a byproduct of such nucleotide incorporation. Optionally, the method further includes detecting the incorporation of the one or more nucleotides by detecting the generation of the one or more hydrogen ions using the FET.

In some embodiments, the detecting includes detecting a change in voltage and/or current at the at least one FET within the array in response to the generation of the one or more hydrogen ions.

In some embodiments, the FET can be selected from the group consisting of: ion-sensitive FET (isFET) and chemically-sensitive FET (chemFET).

One exemplary system involving sequencing via detection of ionic byproducts of nucleotide incorporation is the Ion Torrent PGM™ or Proton™ sequencer (Life Technologies), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™ or Proton™ sequencer detects the nucleotide incorporations by detecting the hydrogen ion byproducts of the nucleotide incorporations. The Ion Torrent PGM™ or Proton™ sequencer can include a plurality of nucleic acid templates to be sequenced, each template disposed within a respective sequencing reaction well in an array. The wells of the array can each be coupled to at least one ion sensor that can detect the release of H⁺ ions or changes in solution pH produced as a byproduct of nucleotide incorporation. The ion sensor comprises a field effect transistor (FET) coupled to an ion-sensitive detection layer that can sense the presence of H⁺ ions or changes in solution pH. The ion sensor can provide output signals indicative of nucleotide incorporation which can be represented as voltage changes whose magnitude correlates with the H⁺ ion concentration in a respective well or reaction chamber. Different nucleotide types can be flowed serially into the reaction chamber, and can be incorporated by the polymerase into an extending primer (or polymerization site) in an order determined by the sequence of the template. Each nucleotide incorporation can be accompanied by the release of H⁺ ions in the reaction well, along with a concomitant change in the localized pH. The release of H⁺ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously. Further details regarding the compositions, design and operation of the Ion Torrent PGM™ or Proton™ sequencer can be found, for example, in U.S. patent application Ser. No. 12/002,781, now published as U.S. Patent Publication No. 2009/0026082; U.S. patent application Ser. No. 12/474,897, now published as U.S. Patent Publication No. 2010/0137143; and U.S. patent application Ser. No. 12/492,844, now published as U.S. Patent Publication No. 2010/0282617, all of which applications are incorporated by reference herein in their entireties.

In a typical embodiment of ion-based nucleic acid sequencing, nucleotide incorporations can be detected by detecting the presence and/or concentration of hydrogen ions generated by polymerase-catalyzed extension reactions. In one embodiment, templates each having a primer and polymerase operably bound can be loaded into reaction chambers (such as the microwells disclosed in Rothberg et al, cited herein), after which repeated cycles of nucleotide addition and washing can be carried out. In some embodiments, such templates can be attached as clonal populations to a solid support, such as particles, bead, or the like, and said clonal populations are loaded into reaction chambers. As used herein, “operably bound” means that a primer is annealed to a template so that the primer's 3′ end may be extended by a polymerase and that a polymerase is bound to such primer-template duplex, or in close proximity thereof so that binding and/or extension takes place whenever nucleotides are added.

In each addition step of the cycle, the polymerase can extend the primer by incorporating added nucleotide only if the next base in the template is the complement of the added nucleotide. If there is one complementary base, there is one incorporation, if two, there are two incorporations, if three, there are three incorporations, and so on. With each such incorporation there is a hydrogen ion released, and collectively a population of templates releasing hydrogen ions changes the local pH of the reaction chamber. The production of hydrogen ions is monotonically related to the number of contiguous complementary bases in the template (as well as the total number of template molecules with primer and polymerase that participate in an extension reaction). Thus, when there are a number of contiguous identical complementary bases in the template (i.e. a homopolymer region), the number of hydrogen ions generated, and therefore the magnitude of the local pH change, can be proportional to the number of contiguous identical complementary bases. If the next base in the template is not complementary to the added nucleotide, then no incorporation occurs and no hydrogen ion is released. In some embodiments, after each step of adding a nucleotide, an additional step can be performed, in which an unbuffered wash solution at a predetermined pH is used to remove the nucleotide of the previous step in order to prevent misincorporations in later cycles. In some embodiments, the after each step of adding a nucleotide, an additional step can be performed wherein the reaction chambers are treated with a nucleotide-destroying agent, such as apyrase, to eliminate any residual nucleotides remaining in the chamber, which may result in spurious extensions in subsequent cycles.

In one exemplary embodiment, different kinds of nucleotides are added sequentially to the reaction chambers, so that each reaction can be exposed to the different nucleotides one at a time. For example, nucleotides can be added in the following sequence: dATP, dCTP, dGTP, dTTP, dATP, dCTP, dGTP, dTTP, and so on; with each exposure followed by a wash step. The cycles may be repeated for 50 times, 100 times, 200 times, 300 times, 400 times, 500 times, 750 times, or more, depending on the length of sequence information desired.

In some embodiments, sequencing can be performed according to the user protocols supplied with the PGM™ or Proton™ sequencer. Example 3 provides one exemplary protocol for ion-based sequencing using the Ion Torrent PGM™ sequencer (Ion Torrent™ Systems, Life Technologies, CA).

### Systems

In some embodiments, the present teachings provide systems for capturing target polynucleotides, comprising any combination of: blocker oligonucleotides, capture oligonucleotides (conjugated or not to a binding moiety), first nucleic acid adaptors, nucleic acid second adaptors, beads (conjugated or not to a binding partner moiety), hybridization solutions, and/or washing solutions. A system can include all or some of these components. In some embodiments, systems for generating reduced complexity target polynucleotides can further comprise any combination of: buffers; cations; size-selection reagents; one or more end-repairing enzyme(s); one or more repairing enzyme(s); one or more nick repair enzymes; one or more ligation enzyme(s); reagents for nucleic acid purification; reagents for nucleic acid amplification; endonuclease(s); polymerase(s); kinase(s); phosphatase(s); and/or nuclease(s).

### Kits

In some embodiments, the present teachings provide kits for capturing target polynucleotides. In some embodiments, kits include any reagent that can be used to capture target polynucleotides from a nucleic acid sample. In some embodiments, kits include any combination of: blocker oligonucleotides, capture oligonucleotides (conjugated or not to a binding moiety), first nucleic acid adaptors, second nucleic acid adaptors, beads (conjugated or not to a binding partner moiety), hybridization solutions, and/or washing solutions. A kit can include all or some of these components. In some embodiments, a kit for generating reduced complexity target polynucleotides can further comprise any combination of: buffers; cations; size-selection reagents; one or more end-repairing enzyme(s); one or more repairing enzyme(s); one or more nick repair enzymes; one or more ligation enzyme(s); reagents for nucleic acid purification; reagents for nucleic acid amplification; endonuclease(s); polymerase(s); kinase(s); phosphatase(s); and/or nuclease(s).

1. A method for capturing target polynucleotides, comprising:
(a) providing a nucleic acid sample having a plurality of non-target polynucleotide constructs which include a plurality of non-target polynucleotides each joined to at least one nucleic acid adaptor, and the nucleic acid sample having a plurality of target polynucleotide constructs which include a plurality of target polynucleotides each joined to at least one nucleic acid adaptor;
(b) contacting the nucleic acid sample with at least one blocker oligonucleotide which hybridizes with the at least one nucleic acid adaptor to reduce non-specific hybridization with the at least one nucleic acid adaptor; and
(c) contacting the nucleic acid sample with at least one capture oligonucleotide which hybridizes to at least a portion of the plurality of target polynucleotides to form at least one capture duplex.

2-5. (canceled)

6. The method of claim 1, wherein the capture oligonucleotide comprises a binding moiety.

7. The method of claim 6, wherein the binding moiety comprises biotin.

8. The method of claim 6 further comprising: binding the binding moiety with a binding partner moiety.

9. The method of claim 8, wherein the binding partner moiety comprises an avidin or streptavidin moiety.

10. The method of claim 8, wherein the binding partner moiety is attached to a bead.

11. The method of claim 10, wherein the bead is magnetic or paramagnetic.

12. The method of claim 11 further comprising: separating the at least one capture duplex, from a plurality of polynucleotide constructs that do not form duplexes to form enriched target polynucleotides.

13. The method of claim 1 further comprising: washing the capture duplexes to remove polynucleotide constructs that do not form duplexes.

14. The method of claim 1, wherein the at least one blocker oligonucleotide hybridizes to at least a portion of the nucleic acid adaptor.

15. The method of claim 1, wherein the at least one adaptor comprises a P1 adaptor sequence according to the nucleotide sequence of SEQ ID NOS:3 or 5.

16. The method of claim 1, wherein the at least one adaptor comprises an A adaptor sequence according to the nucleotide sequence of SEQ ID NOS:140 or 141.

17. The method of claim 1, wherein the at least one blocker oligonucleotide comprises a P1 sequence according to the nucleotide sequence of SEQ ID NO:112 or 139.

18. A capture duplex produced by the method of claim 1.

19. An enriched target polynucleotide produced by the method of claim 12.

20. The method of claim 12, further comprising sequencing the enriched target polynucleotide.
